(19) United States
(12) Patent Application Publication
(10) Pub. No.: US 2007/0179263 A1
(43) Pub. Date: Aug. 2, 2007

(54) Functional material comprising fluorine-containing compound

(75) Inventors: Meiten Koh, Osaka (JP); Mamoru Miyata, Osaka (JP); Takayuki Araki, Osaka (JP)

Correspondence Address: SUGHRUE MION, PLLC, 2100 PENNSYLVANIA AVENUE, N.W., SUITE 800, WASHINGTON, DC, 20037, US

(73) Assignee: DAIKIN INDUSTRIES LTD., OSAKA-SHI, OSAKA (JP)

(21) Appl. No.: 10/591,860

(22) PCT Filed: Mar. 3, 2005
(86) PCT No.: PCT/JP05/03611

§ 371 Date: Sep. 6, 2006

(30) Foreign Application Priority Data
Mar. 8, 2004 (JP) 2004-064826
Jun. 11, 2004 (JP) 2004-174434

(51) Int. Cl. C08F 214/18; C07C 215/28

(52) U.S. Cl. 526/242; 526/247; 526/248; 564/505; 564/360

(57) ABSTRACT

There is provided an ionic liquid type functional material which is useful for a lubricant, acid-removing agent, various ionic liquid materials, electrolyte for solar cell and actuator material. The ionic liquid type functional material contains an aromatic compound which has a fluorine-containing ether chain and is represented by the formula (1):
[Ra-D [symbol] ₘRy (1) wherein -D- is a fluoroether unit represented by the formula (1-1): [symbol]
O—R [symbol] ₙ or [symbol] R—O [symbol] ₙ (1-1)
in which R is at least one selected from divalent fluorine-containing alkylene groups having 1 to 5 carbon atoms in which at least one of hydrogen atoms is replaced by fluorine atom; n is an integer of from 1 to 20; when m is not less than 2, two or more of D may be the same or different; Ra is a monovalent organic group which has 1 to 20 carbon atoms and does not contain D; when m is not less than 2, two or more of Ra may be the same or different; m is an integer of from 1 to 4; Ry is a mono-, di-, tri- or tetra-valent organic group having 2 to 30 carbon atoms which has at least one selected from a basic functional group Y¹ and/or a salt Y² of the basic functional group and contains an aromatic ring structure, provided that a unit of —O—O— is not contained in the formulae (1) and (1-1).

### TECHNICAL FIELD

The present invention relates to a functional material comprising an aromatic compound which has a basic functional group and contains a structural unit derived from a specific fluorine-containing ether. This functional material is useful as an ionic liquid, electrolyte for solar cell, lubricant, acid-removing agent, actuator material and the like.

The present invention further relates to a functional material comprising a fluorine-containing polymer which has, in its side chain, a moiety of aromatic ring structure having a basic functional group and a moiety of fluorine-containing ether structure. This functional material is useful as an ionic liquid type polymer.

### Background Art

A fluorine-containing ether has been so far used mainly in the form of polyether for various applications such as a lubricant and grease as an oil having high thermal stability and chemical stability by taking advantage of its excellent oxidation resistance, weather resistance and chemical resistance. Also a fluorine-containing polyether having at its one end or both ends carboxylic acid, sulfonic acid, hydroxyl group or acid salt by neutralizing such an acid with a base is used for applications such as an antireflection film for resist and a material for protecting a substrate by taking advantage of its properties of enhancing solubility in a solvent and enhancing adhesion to a substrate by introducing a polar group. Also a fluorine-containing polyether in which one end or both ends thereof are converted to neutral functional groups such as acryloyl groups or alkoxysilyl groups is used for antireflection film application and oxygen-enriched membrane application by taking advantage of its property of being curable by using such functional groups as a crosslinking site.

On the other hand, fluorine-containing ether compounds having functional group showing basicity have been hardly known. There are a report that a long chain fluorine-containing polyether having aromatic amide is used for oil application (JP47-1895A), and a report that alkoxy phosphite is contained in the same polyether as mentioned above similarly for use for oil application (JP1-265049A). However in those reports, it is not considered that a functional group showing basicity is contained positively to take advantage of its properties, and it is considered that containing a basic functional group should be avoided as mentioned in Comparative Example of JP47-1895A. Such being the case, unlike the fluorine-containing ether compounds having acidic or neutral functional group, attention has hardly been directed to fluorine-containing ether compounds having basic functional group, and compounds having basic functional group and being applicable to various functional materials have not yet been found.

Ionic liquids are molten salts being in the form of liquid within a temperature range of from room temperature to relatively high temperatures (to 300° C.), and have the following characteristics.

(a) Generally ionic liquids exhibit high polarity and high dissolving power for low molecular weight organic and inorganic compounds.

(b) Because of a very low vaporization pressure and non-volatility, when used as a medium for synthesis reaction, ionic liquids can be used in vacuo and can provide a clean reaction environment.

(c) Since ionic liquids show insolubility in some organic solvents and water, a reaction environment in a two-layer medium can be provided, thereby separation between a starting material and a product is made easy, and control of interface reaction is made easy, for example, control of stereoselectivity of a product of synthesis reaction can be made, and thus various novel organic synthesis reactions have been provided.

So far various imidazolium salt compounds have been investigated as an ionic liquid.

For example, salts of N,N′-dialkylimidazolium cation with tetrafluoroborate anion (BF₄⁻) and hexafluorophosphate anion (PF₆⁻) and the like have been investigated for various applications as an ionic liquid having stability in water.

However those ionic liquids (salt of N-methylimidazolium or N′-butylimidazolium with hexafluorophosphoric acid) are high in a viscosity, and when used as a medium for synthesis reaction, has a problem that diffusion of a solute is difficult to occur. Also there is a problem that those conventional ionic liquids cannot dissolve synthetic polymers such as polyvinyl alcohol, biopolymers such as protein, polysaccharide and nucleic acid, and molecular aggregates such as micelle and bimolecular film, and functions derived therefrom cannot be exhibited sufficiently.

In order to solve those problems attributable to a high viscosity, there is proposed an ionic liquid obtained by introducing alkoxyl group to an alkyl moiety of N,N′-dialkylimidazolium salt (JP2002-3478A), and it is reported that a viscosity can be decreased and synthetic polymers, biopolymers and molecular aggregates can be dissolved.

However the above-mentioned N,N′-dialkylimidazolium salt and N,N′-dialkylimidazolium salt possessing introduced alkoxyl group (JP2002-3478A) have a problem that a viscosity is high and an effect of decreasing a viscosity is insufficient even by introduction of alkoxyl group, and further those ionic liquids are insufficient in stability of heat resistance and oxidation resistance and durability.

Also on the other hand, there are disclosed, as an ionic liquid type polymer, N-vinylimidazolium salt polymers obtained by polymerizing N-vinylimidazolium or N′-alkylimidazolium salt (JP2000-11753A). N-Vinylimidazolium salt which is a monomer is in the form of ionic liquid, but a polymer having an imidazolium structure which is obtained by polymerization of N-vinylimidazolium salt is a solid and is not in the form of liquid at normal temperature.

In the light of the present situation mentioned above, an object of the present invention is to provide a functional material which is prepared by using an aromatic compound having a low viscosity, heat resistance and oxidation resistance and is useful as an ionic liquid. Concretely an object of the present invention is to provide a material exhibiting functions as an ionic liquid, namely a functional material which is useful, for example, for synthetic reaction medium, extraction medium, acid-removing agent, electrolyte for solar cell, lubricant, actuator material and the like.

Another object of the present invention is to provide a novel aromatic compound and novel fluorine-containing polymer which are usable for the above-mentioned ionic liquid type functional material.

### DISCLOSURE OF INVENTION

The present inventors have made intensive studies with respect to compounds having basic functional group and as a result, have found that a specific aromatic compound having a specific fluorine-containing ether chain and basic functional group shows a good low viscosity and is excellent in heat resistance and oxidation resistance.

Further the present inventors have made intensive studies also with respect to polymers having basic functional group and as a result, have found that a polymer having a specific basic functional group and a specific fluorine-containing ether structure in the same side chain thereof is in good liquid state and is excellent in heat resistance and oxidation resistance.

The present inventors also have found that those aromatic compound and polymer have excellent performance as a compound constituting an ionic liquid type functional material, for example, a highly functional material such as an ionic liquid, electrolyte for solar cell, actuator, lubricant, acid-removing agent or the like, and have completed the present invention.

The present invention relates to an ionic liquid type functional material containing an aromatic compound which has a fluorine-containing ether chain and is represented by the formula (1):
[Ra-D [symbol] ₘRy (1)
wherein -D- is a fluoroether unit represented by the formula (1-1): [symbol]
O—R [symbol] ₙ or [symbol] R—O [symbol] ₙ (1-1)
in which R is at least one selected from divalent fluorine-containing alkylene groups having 1 to 5 carbon atoms in which at least one of hydrogen atoms is replaced by fluorine atom; n is an integer of from 1 to 20, and when m is not less than 2, two or more of D may be the same or different;
Ra is a monovalent organic group which has 1 to 20 carbon atoms and does not contain the above-mentioned D, and when m is not less than 2, two or more of Ra may be the same or different; m is an integer of from 1 to 4;
Ry is a mono-, di-, tri- or tetra-valent organic group having 2 to 30 carbon atoms which has at least one selected from basic functional groups Y¹ and/or salts Y² of the basic functional groups and contains an aromatic ring structure,
   provided that a unit of —O—O— is not contained in the formulae (1) and (1- 1).

It is preferable that —O—R— in -D- is at least one fluoroether unit selected from the group consisting of —(OCFZ¹CF₂)—, —(OCF₂CF₂CF₂)—, —(OCH₂CF₂CF₂)—, —(OCFZ²)-, —(OCZ³₂)-, —(CFZ¹CF₂O)—, —(CF₂CF₂CF₂O)—, —(CH₂CF₂CF₂O)—, —(CFZ²O)— and —(CZ³₂O)—, wherein Z¹ and Z² are the same or different and each is H, F or CF₃; Z³ is CF₃.

It is preferable that Ra is selected from fluorine-containing alkyl groups Rx having 1 to 20 carbon atoms.

It is preferable that the basic functional group or the salt of the basic functional group which Ry has is at least one selected from amines, imines, enamines, ketimines, azines and salts thereof.

Also the present invention relates to an ionic liquid type functional material containing a fluorine-containing polymer represented by the formula (M-1):
-(M1)-(A1)- (M-1)
wherein the structural unit M1 is at least one selected from structural units derived from ethylenic monomers having, in a side chain thereof, a moiety represented by the formula (2):
-D¹-Ry¹ (2)
in which -D¹- is a fluoroether unit represented by the formula (2-1): [symbol]
O—R¹ [symbol] ₙ₁ or [symbol] R¹—O [symbol] ₙ₁ (2-1)
wherein R¹ is at least one selected from divalent fluorine-containing alkylene groups having 1 to 5 carbon atoms in which at least one of hydrogen atoms is replaced by fluorine atom; n1 is an integer of from 1 to 20; Ry¹ is a monovalent organic group having 2 to 30 carbon atoms which has at least one selected from basic functional groups Y¹ and/or salts Y² of the basic functional groups and contains an aromatic ring structure, provided that a unit of —O—O— is not contained in the structural unit M1 and the formula (2-1); the structural unit A1 is a structural unit derived from a monomer being copolymerizable with the monomer being capable of providing the structural unit M1, and the structural units M1 and A1 are contained in amounts of from 1 to 100% by mole and from 0 to 99% by mole, respectively.

It is preferable that the basic functional group Y¹ or the salt Y² of the basic functional group which is contained in Ry is at least one selected from amines, imines, enamines, ketimines, azines and salts thereof.

Also the present invention relates to a novel aromatic compound which has a fluorine-containing ether chain and is represented by the formula (4):
[Rx²-D² [symbol] ₘ₂Ry³ (4)
wherein -D²- is a fluoroether unit represented by the formula (4-1): [symbol]
O—R² [symbol] ₙ₂ or [symbol] R²—O [symbol] ₙ₂ (4-1)
in which R² is at least one selected from divalent fluorine-containing alkylene groups having 1 to 5 carbon atoms in which at least one of hydrogen atoms is replaced by fluorine atom; n2 is an integer of from 1 to 20, and when m2 is not less than 2, two or more of D² may be the same or different; Ry³ is a mono-, di-, tri- or tetra-valent organic group having 2 to 30 carbon atoms which has at least one selected from amines and/or salts of amines and contains an aromatic ring structure; Rx² is a fluorine-containing alkyl group having 1 to 20 carbon atoms, and when m2 is not less than 2, two or more of Rx² may be the same or different; m2 is an integer of from 1 to 4, provided that a unit of —O—O— is not contained in the formulae (4) and (4-1).

Further the present invention relates to a novel aromatic compound which has a fluorine-containing ether chain and is represented by the formula (5):
CX¹X²═CX³—(CX⁴X⁵)ₙ₃(C═O)ₙ₄-D²-Ry⁴ (5)
wherein X¹, X², X⁴ and X⁵ are the same or different and each is hydrogen atom or fluorine atom; X³ is selected from hydrogen atom, fluorine atom, CH₃ and CF₃; n3 and n4 are the same or different and each is 0 or 1; Ry⁴ is a monovalent organic group having 2 to 30 carbon atoms which has at least one selected from amines and/or salts of amines and contains an aromatic ring structure; D² is as defined in the formula (4).

Also the present invention relates to a novel fluorine-containing polymer, which has a number average molecular weight of from 500 to 1,000,000 and is represented by the formula (M-3):
-(M3)-(A3)- (M-3)
wherein the structural unit M3 is a structural unit represented by the formula (7): 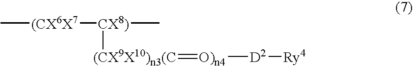
wherein X⁶, X⁷, X⁹ and X¹⁰ are the same or different and each is hydrogen atom or fluorine atom; X⁸ is selected from hydrogen atom, fluorine atom, CH₃ and CF₃; n3 and n4 are the same or different and each is 0 or 1; D² and Ry⁴ are as defined in the formula (5); the structural unit A3 is a structural unit derived from a monomer being copolymerizable with the monomer being capable of providing the structural unit M3, and the structural units M3 and A3 are contained in amounts of from 1 to 100% by mole and from 0 to 99% by mole, respectively.

### BEST MODE FOR CARRYING OUT THE INVENTION

The ionic liquid type functional material of the present invention is prepared by using an aromatic compound which has a fluorine-containing ether chain and is represented by the formula (1):
[Ra-D [symbol] ₘRy (1)
wherein -D- is a fluoroether unit represented by the formula (1-1): [symbol]
O—R [symbol] ₙ or [symbol] R—O [symbol] ₙ (1-1)
in which R is at least one selected from divalent fluorine-containing alkylene groups having 1 to 5 carbon atoms in which at least one of hydrogen atoms is replaced by fluorine atom; n is an integer of from 1 to 20, and when m is not less than 2, two or more of D may be the same or different;
Ra is a monovalent organic group which has 1 to 20 carbon atoms and does not contain the above-mentioned D, and when m is not less than 2, two or more of Ra may be the same or different;
m is an integer of from 1 to 4;
Ry is a mono-, di-, tri- or tetra-valent organic group having 2 to 30 carbon atoms which has at least one selected from basic functional groups Y¹ and/or salts Y² of the basic functional groups and contains an aromatic ring structure,
provided that a unit of —O—O— is not contained in the formulae (1) and (1-1).

The first feature of the aromatic compound used for the ionic liquid type functional material is to contain the fluorine-containing ether unit represented by -D- in the above-mentioned formula (1), concretely 1 to 20 repeat units of —(O—R)— or —(R—O)—.

—R—is a divalent fluorine-containing alkylene group having 1 to 5 carbon atoms and has at least one fluorine atom, whereby as compared with a group having a non-fluorine-containing alkoxyl group or non-fluorine-containing alkylene ether unit, the aromatic compound intended to be used for an ionic liquid can be liquefied or a viscosity of the aromatic compound can be further decreased.

Further the fluorine-containing ether unit -D- is preferred also because heat resistance and oxidation resistance can be greatly improved.

The higher the fluorine content of R in the fluorine-containing ether unit -D-, the higher its effect on reduction of a viscosity and on heat resistance and oxidation resistance. The fluorine content of fluorine-containing alkylene group R is preferably from 45 to 76% by mass, more preferably from 55 to 76% by mass, especially preferably from 65 to 76% by mass, and most preferably R is a perfluoroalkylene group (76% by mass).

Examples of —(O—R)— or —(R—O)— in -D- are concretely —(OCF₂CF₂CF₂)—, —(CF₂CF₂CF₂O)—, —(OCFZ¹CF₂)—, —(OCF₂CFZ¹)-, —(OCFZ²)-, —(CFZ²O)—, —(OCH₂CF₂CF₂)—, (OCF₂CF₂CH₂)—, —(OCH₂CH₂CF₂)—, —(OCF₂CH₂CH₂)—, —(OCF₂CF₂CF₂CF₂)—, —(CF₂CF₂CF₂CF₂O)—, —(OCFZ²CH₂)—, —(CH₂CFZ²O)—, —(OCH(CH₃)CF₂CF₂)—, —(OCF₂CF₂CH(CH₃))—, —(OCZ³₂)- and —(CZ³₂O)— wherein Z¹ and Z² are the same or different and each is H, F or CF₃; Z³ is CF₃. It is preferable that -D- is at least one of those repeat units.

Among them, it is preferable that -D- is at least one repeat unit selected from —(OCFZ¹CF₂)—, —(OCF₂CF₂CF₂)—, —(OCH₂CF₂CF₂)—, —(OCFZ²)-, —(OCZ³₂)- —(CFZ¹CF₂O)—, —(CF₂CF₂CF₂O)—, —(CH₂CF₂CF₂O)—, —(CFZ²O)— and —(CZ³₂O)—. It is particularly preferable that -D- is at least one repeat unit selected from —(OCFZ¹CF₂)—, —(OCF₂CF₂CF₂)—, —(OCH₂CF₂CF₂)—, —(CFZ¹CF₂O)—, —(CF₂CF₂CF₂O)— and —(CH₂CF₂CF₂O)—, further preferably at least one repeat unit selected from —(OCFZ¹CF₂)—, —(OCF₂CF₂CF₂)—, —(CFZ¹CF₂O)— and —(CF₂CF₂CF₂O)—.

A structural unit of —O—O— (concretely —R—O—O—R—, —O——R—, —R—O—O— or the like) is not contained in the above-mentioned fluorine-containing ether unit -D- and in the aromatic compound of the formula (1).

Those preferred fluorine-containing ethers, particularly perfluoro ethers can effectively liquefy the aromatic compound intended for an ionic liquid and can decrease a viscosity thereof and further can provide an ionic liquid type functional material having higher heat resistance and oxidation resistance.

The number n of repeated fluorine-containing ether units in -D- is optionally selected depending on purpose, intention and application and is selected from the number of repeat units of from 1 to 20.

The larger number n functions for decrease in a viscosity more effectively, but the number n exceeding 20 is not preferred because an effect on basicity and dielectric constant derived from the aromatic ring moiety Ry containing basic functional group explained infra is lowered.

The effect on basicity and dielectric constant which is derived from Ry of the compound of the present invention is important for applications to an ionic liquid, actuator and electrolyte for solar cell explained infra. In those cases, the number n of repeated fluorine-containing ether units is preferably from 1 to 15, further preferably from 1 to 12.

For example, for removing an acid from waste water, there is a case where an anionic polymer is used as an acid-removing agent from the viewpoint of easy separation from water and easy recycling. However since an acid-removing process is a liquid-solid reaction, an acid-removing efficiency cannot always be said to be good. The compound of the present invention containing a fluorine-containing ether is a liquid, and therefore an acid-removing process is a liquid-liquid reaction which is high in efficiency. But in the case of a short fluorine-containing ether chain, there is a case where the compound is dissolved in the acidic aqueous solution and separation thereof becomes difficult. From this point of view, a longer fluorine-containing ether chain is preferred.

Therefore in the case of applications to materials such as an acid-removing agent and lubricant, in which the features of the fluorine-containing ether unit are utilized, the larger the number n of repeated fluorine-containing ether units, the more preferable, namely the number of repeat units is from 5 to 20, more preferably from 10 to 20.

The second feature of the aromatic compound of the formula (1) used for the ionic liquid type functional material of the present invention is that the moiety Ry containing the basic functional group and having an aromatic ring structure is bonded to the fluorine-containing ether moiety -D-. To Ry is bonded at least one moiety containing the fluorine-containing ether -D-, and 2 to 4 moieties may be bonded to Ry.

Ry is a mono-, di-, tri- or tetra-valent organic group having 2 to 30 carbon atoms which has at least one selected from the basic functional groups Y¹ and/or the salts Y² of the basic functional groups and contains an aromatic ring structure.

The basic functional group Y¹ in Ry may be selected from functional groups having a pKa value larger than that of water, and is usually selected from functional groups having a pKa value of not less than 20. The pKa value is preferably not less than 20, more preferably not less than 25, particularly preferably not less than 28.

A too low basicity (a too low pKa value) is not preferred because an ionic function as an ionic liquid is not exhibited sufficiently when forming into a salt by reaction with an acid.

For example, when the compound is used as a reaction medium or as a part thereof, dissolution of a targeted reaction compound becomes difficult, an intended reaction does not occur and selectivity of a targeted reaction product cannot be obtained. Also in the case of use as an acid-removing agent, a targeted acid cannot be trapped or separated and extracted. In the case of use for actuator application, intended moving performance cannot be obtained.

Concretely the basic functional group Y¹ in Ry is preferably at least one selected from phosphoric acid amides, phosphoric acid imides, amines, imines, enamines, ketimines, hydroxylamines, amidines, azines, hydrazines, oximes and amine oxides, more preferably one selected from amines, imines, enamines, ketimines and azines, and particularly preferably amines.

When amine is bonded as a substituent to the carbon atom of aromatic ring as explained infra, any of primary amino group (—NH₂), secondary amino group (—NR¹H) and tertiary amino group (—NR²R³) can be used. Usually also in the case of secondary amino group and tertiary amino group, preferred is a lower amino group having a hydrocarbon group (R¹, R² or R³) having smaller number of carbon atoms from the viewpoint of oxidation resistance. For example, there are hydrocarbon groups having 1 to 10 carbon atoms, preferably hydrocarbon groups having 1 to 5 carbon atoms, more preferably methyl or ethyl. Particularly preferred from the viewpoint of oxidation resistance is a primary amino group (—NH₂).

When a nitrogen atom of an amino group constitutes an aromatic ring at the same time, usually a form of secondary or tertiary amino group can be taken. Those cyclic amino groups themselves are more excellent in oxidation resistance, and when a monovalent hydrocarbon group is further bonded to the nitrogen atom of the cyclic amino group, hydrocarbon groups having smaller number of carbon atoms are preferred in the same manner as mentioned above. For example, there are hydrocarbon groups having 1 to 10 carbon atoms, preferably hydrocarbon groups having 1 to 5 carbon atoms, more preferably methyl or ethyl. Preferred most from the viewpoint of oxidation resistance is a cyclic amino group in which a monovalent hydrocarbon group is not bonded to the nitrogen atom.

In the ionic liquid type functional material of the present invention, it is preferable that the functional group contained in Ry is the salt Y² of the basic functional group or contains the salt Y² of the basic functional group.

The salt Y² of the basic functional group is not limited particularly as far as it is a salt of the above-mentioned basic functional group Y¹ and an anionic ion (anion species), and is optionally selected depending on kind, function, purpose and application of the aromatic compound.

Also the salt Y² of the basic functional group may be a salt obtained by subjecting the basic functional group Y¹ to cationic quaternization, for example, salts of quaternary ammonium cation obtained by quaternizing amines.

Examples of the anions in the salt Y² of the basic functional group are, for instance, anions derived from halogen atoms such as Cl⁻, Br⁻ and F⁻; anions derived from inorganic acids such as HSO₃⁻, NO₃⁻, ClO₄⁻, PF₆⁻, BF₄⁻ and SbF₆⁻; and anions derived from organic acids such as CF₃SO₃⁻, ⁻N(SO₂CF₃)₂, ⁻C(SO₂CF₃)₂ and ⁻OCOCF₃.

Among them, anions preferred for an ionic liquid type functional material from the viewpoint of making liquefaction easy and making it possible to decrease a viscosity are those selected from the group consisting of anions derived from inorganic acids and anions derived from organic acids, and particularly preferred are anions derived from inorganic acids.

Further preferred are anions being capable of forming Lewis acid, for example, PF₆⁻, BF₄⁻, SbF₆⁻ and the like.

In the ionic liquid type functional material of the present invention, Ry is featured by having an aromatic ring structure in addition to the above-mentioned basic functional group Y¹ and the salt Y² of the basic functional group.

The moiety of aromatic ring structure may be one having an aromatic ring structure formed by using only carbon atoms, one having an aromatic ring structure formed by using carbon atoms and hetero atoms such as nitrogen atom, sulfur atom or oxygen atom or one having a monocyclic structure or polycyclic structure (fused ring).

Examples of the moiety of aromatic ring structure are: 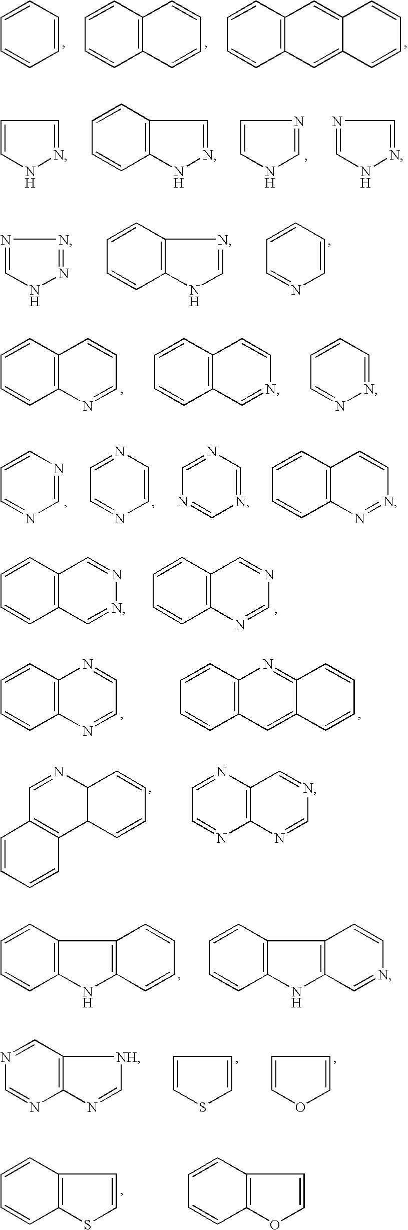
and the like.

The presence of those aromatic ring structures is preferred because enhancement of oxidation resistance and improvement in a dielectric constant are given to the ionic liquid type functional material of the present invention.

In the ionic liquid type functional material of the present invention, the presence of the fluorine-containing ether unit -D- is usually disadvantageous from the viewpoint of a dielectric constant, but by introducing the aromatic ring structure, a dielectric constant can be improved.

Concretely improvement of a dielectric constant is particularly preferred in ionic liquid application and solar cell electrolyte application.

Also the presence of the aromatic ring structure is preferred since interaction (affinity) with various inorganic compounds, hydrocarbon compounds and polymer compounds is high and adsorption property can be improved. In the ionic liquid type functional material of the present invention, the presence of the fluorine-containing ether unit -D- is usually disadvantageous from the viewpoint of adsorption, but by introducing the aromatic ring structure, adsorption with various inorganic and organic materials can be improved. By this improvement of adsorption, the functional material can be a material suitable for lubricant application and the like requiring adhesion to a substrate.

The basic functional group Y¹ or the salt Y² of the basic functional group (hereinafter Y¹ and Y² are collectively referred to as functional group Y) may be bonded to the aromatic ring structure as a substituent outside the aromatic ring structure, or the hetero atom forming the functional group Y (cation portion in the case of a salt) may constitute the aromatic ring at the same time.

In the case of the bonding of the functional group Y to the aromatic ring structure as a substituent, the functional group Y may be bonded directly to the carbon atom of the aromatic ring, or the functional group Y may be bonded to the carbon atom of the aromatic ring via a bond (spacer) of divalent organic group.

It is preferable that the divalent organic group which is the bond is selected from divalent hydrocarbon groups having 1 to 10 carbon atoms, more preferably divalent alkylene groups having 1 to 5 carbon atoms, particularly preferably methylene or ethylene. Hydrocarbon groups having a too long chain are not preferred because oxidation resistance is lowered.

Further the functional group Y may be contained as —Y— in the substituent of the aromatic ring.

In the case of forming an aromatic ring structure by using carbon atom and hetero atom, the above-mentioned hetero atom forming the functional group Y (cation portion) may constitute the aromatic ring at the same time.

Also as mentioned above, the functional group Y may be one or more and is present as a substituent of the aromatic ring structure or is present in the ring structure.

Concretely there are following aromatic ring structures.

(i) Aromatic Ring Structures in Which the Functional Group Y is Bonded to the Carbon Atom of the Aromatic Ring as a Substituent

Examples thereof are: 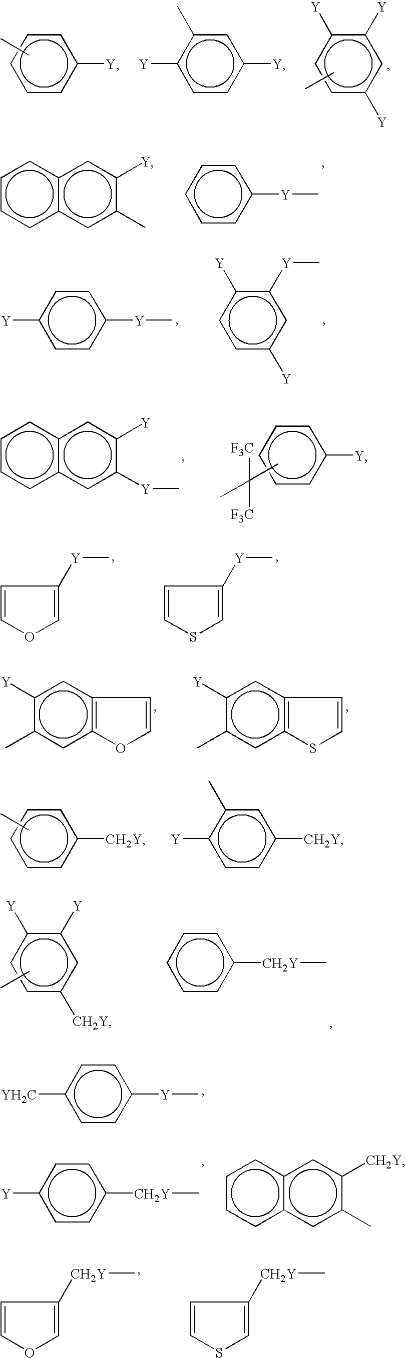
and the like.
(ii) Aromatic Ring Structures in Which the Atom Forming the Functional Group Y (Cation Portion in the Case of the Salt Y²) Constitutes the Aromatic Ring at the Same Time

Examples thereof when the functional group Y is amine are: 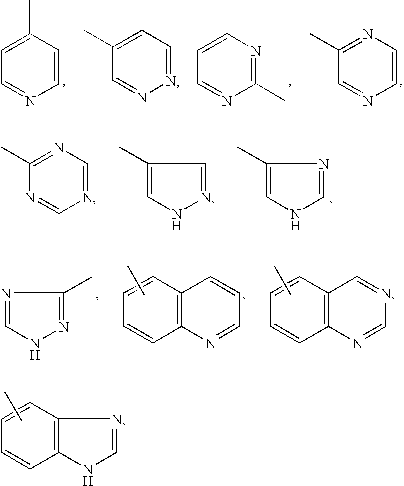
and the like.
(iii) Aromatic Ring Structures in Which the Functional Group Y is Further Bonded as a Substituent to the Carbon Atom of the Aromatic Ring Structure (ii)

Preferred examples thereof are: 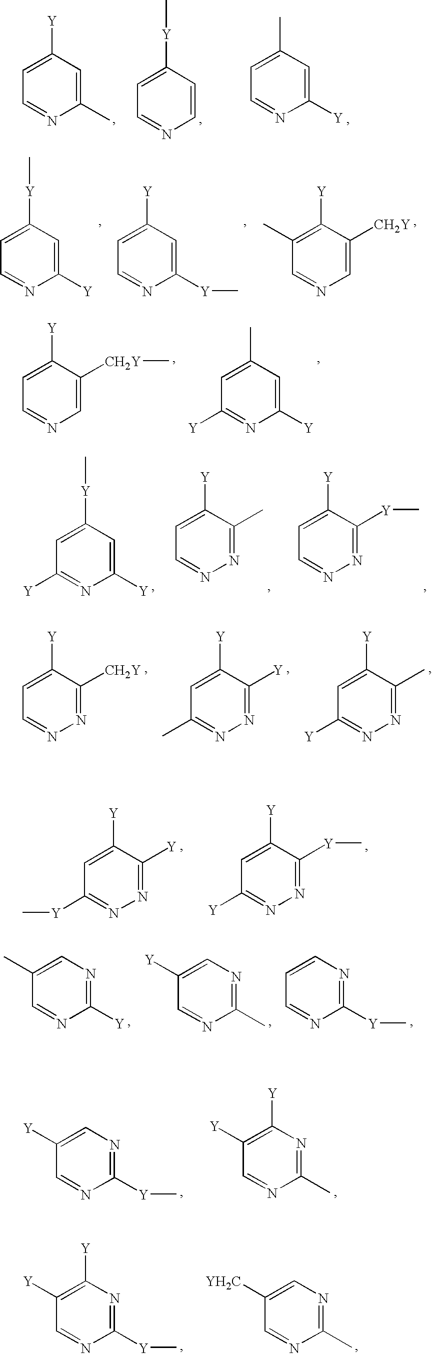 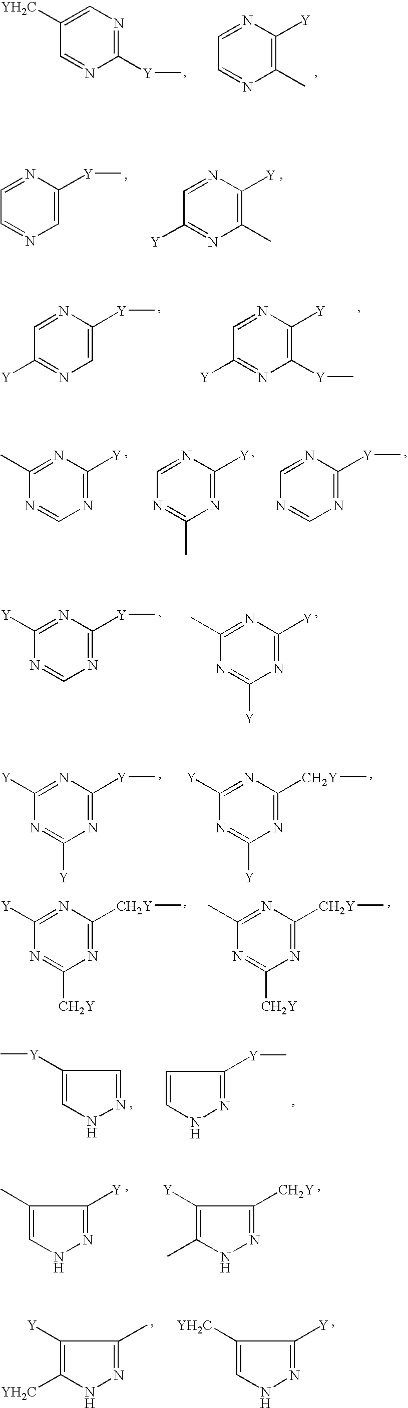 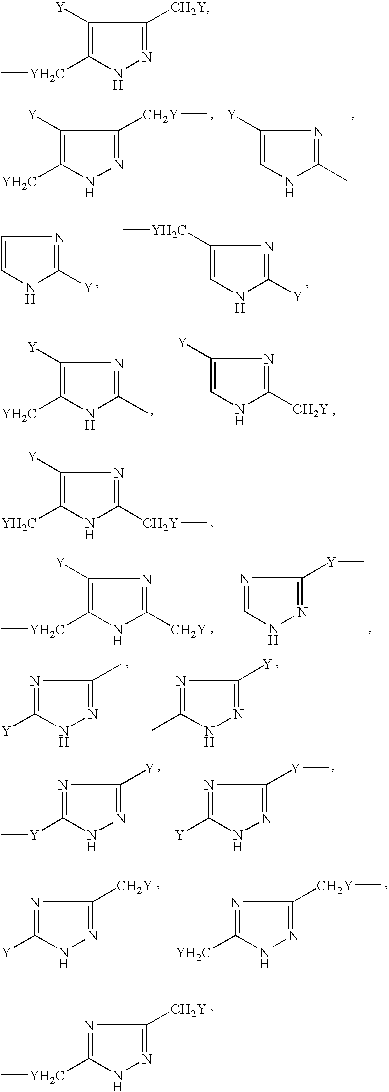
and the like.

Among the moieties of the above-mentioned aromatic ring structures (i) to (iii), preferred are the aromatic ring structures (ii) in which the atom forming the functional group Y (cation portion) constitutes the aromatic ring at the same time and the aromatic ring structures (iii) subjected to replacement by the functional group because an amount (concentration) of functional groups can be increased, and also because it is possible to enhance functions as an ionic liquid type functional material, a dissolving power of a solute and ability of acid trap in the case of a reaction medium and extraction medium, and moving performance in the case of an actuator.

Also those moieties (ii) and (iii) are preferred from the viewpoint of oxidation resistance.

Further among the ring structures of the moiety Ry of the aromatic ring structures of the present invention, monocyclic structures are particularly preferred and can decrease a viscosity and undergo liquefaction effectively.

In the compound of the formula (1) to be used for the ionic liquid type functional material of the present invention, the first bonding of the organic group Ry containing the aromatic ring structure and having the functional group Y to the moiety -D- of the fluorine-containing ether structure is the direct bonding of Ry exemplified in (i), (ii) and (iii) above to -D-, and the second one is the bonding via a bond (spacer) being provided between Ry and -D-. When Ry and -D- are bonded via the bond, it is assumed that the bond (-A-) is included in Ry (when Ry has the bond -A-, Ry is represented by -A-Ry″).

The direct bonding of Ry to -D- is preferred particularly from the viewpoint of heat resistance and oxidation resistance.

When bonding via the bond, the bond (-A-) is not limited particularly as far as it is a divalent bond with covalent bonding. The bond (-A-) is selected from divalent hetero atoms and divalent organic groups.

More preferred are ether bond (—O—), thioether bond (—S—), divalent alkylene group, divalent fluorine-containing alkylene group, ester bond, sulfonic ester bond, phosphoric ester bond, acid amide bond, amidine bond and the like from the point of not lowering oxidation resistance.

(a) In the Case of the Bond -A- Being a Divalent Alkylene Group or Divalent Fluorine-containing Alkylene Group

The divalent alkylene group is preferably selected from divalent alkylene groups having 1 to 10 carbon atoms, and is more preferably a divalent alkylene group having 1 to 5 carbon atoms, particularly preferably methylene group or ethylene group. Hydrocarbon groups having a too long chain is not preferred because oxidation resistance is lowered.

The divalent fluorine-containing alkylene group is the above-mentioned alkylene group in which a part or the whole of hydrogen atoms are replaced by fluorine atoms. From the viewpoint of oxidation resistance, replacement by more fluorine atoms is preferred, and particularly preferred are perfluoro alkylene groups.

Preferred examples thereof are: 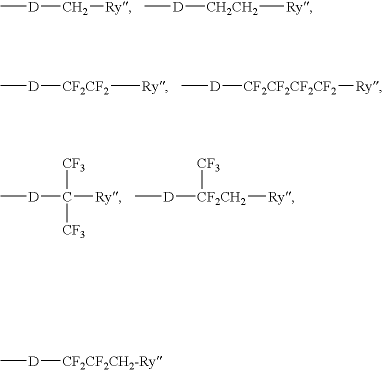
and the like.

Those bonds are preferred from the viewpoint of oxidation resistance.

(b) In the Case of the Bond -A- Being an Ether Bond (—O—) or Thioether Bond (—S—)

Concretely there are -D-O-Ry″, -D-S-Ry″ and the like.

Particularly preferred is an ether bond from the viewpoint of oxidation resistance, heat resistance and chemical resistance.

(c) In the Case of the Bond -A- Being an Ester Bond, Sulfonic Ester Bond or Phosphoric Ester Bond

Examples thereof are: 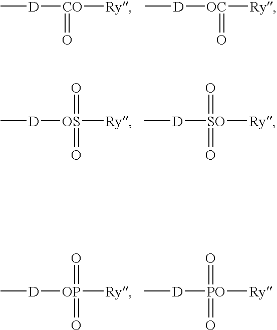
and the like.
(d) In the Case of the Bond -A- Being an Amide Bond

Concretely there are: 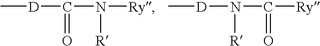
and the like, wherein R′ is hydrogen atom or a hydrocarbon group having 1 to 10 carbon atoms, and from the point of not lowering oxidation resistance and heat resistance, it is preferable that R′ is hydrogen atom or an alkyl group having 1 to 5 carbon atoms, particularly preferably hydrogen atom or methyl.

Examples thereof are: 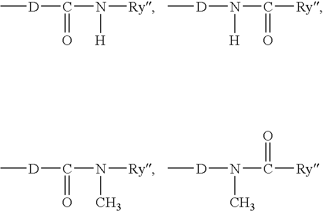
and the like.

Those bonds are preferred from the point that oxidation resistance and heat resistance are excellent.

(e) In the Case of the Bond -A- Being a Sulfonic Acid Amide Bond or Phosphoric Acid Amide Bond

Concretely there are preferably: 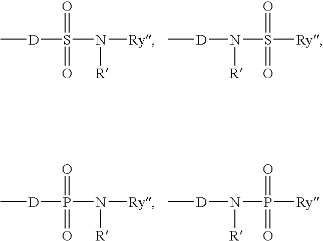
and the like, wherein R′ is hydrogen atom or a hydrocarbon group having 1 to 10 carbon atoms.

From the point of not lowering oxidation resistance and heat resistance, it is preferable that R′ is hydrogen atom or an alkyl group having 1 to 5 carbon atoms, particularly preferably hydrogen atom or methyl.

Preferred examples thereof are: 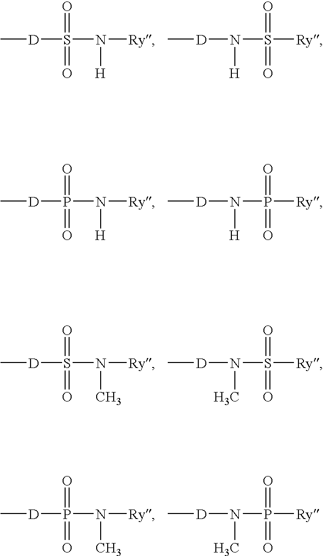
and the like.
(f) in the Case of the Bond -A- Being an Amidine Bond

Concretely there are preferably: 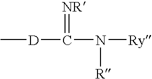
and the like, wherein R′ and R″ are the same or different and each is hydrogen atom or a hydrocarbon group having 1 to 10 carbon atoms.

From the point of not lowering oxidation resistance and heat resistance, it is preferable that R′ and R″ are hydrogen atom or an alkyl group having 1 to 5 carbon atoms, particularly preferably hydrogen atom or methyl.

Preferred examples thereof are: 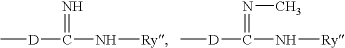
and the like.

Among those bonds exemplified in (a) to (f), a divalent alkylene group, divalent fluorine-containing alkylene group, ether bond, amide bond and amidine bond are preferred particularly from the viewpoint of oxidation resistance.

Also in Ry exemplified in (a) to (f), examples of Ry exemplified supra in (i), (ii) and (iii) can be preferably used similarly as the examples of the moiety Ry″ of the aromatic ring structure having the functional group Y.

In the aromatic compound of the formula (1) to be used for the ionic liquid type functional material of the present invention, Ra is a residue bonded to another end of -D- and is not limited particularly as far as it is selected from monovalent organic groups which have 1 to 20 carbon atoms and do not contain the structure -D-.

Preferred examples of Ra are (iv) fluorine-containing alkyl groups Rx which have 1 to 20 carbon atoms and may have ether bond.

The introduction of the Rx is preferred because the aromatic compound (1) can have a low viscosity and can be liquefied and also because oxidation resistance can be imparted to the aromatic compound.

The fluorine-containing alkyl group Rx has preferably 1 to 10 carbon atoms, more preferably 1 to 5 carbon atoms, which is preferred from the viewpoint of decrease in a viscosity, liquefaction and oxidation resistance.

Further the fluorine-containing alkyl group Rx having a higher fluorine content is preferred from the viewpoint of oxidation resistance. The fluorine content is not less than 40% by mass, more preferably not less than 50% by mass, particularly preferably not less than 60% by mass, and most preferably Rx is a perfluoro alkyl group.

Concretely examples thereof are:
C₃F₇—, CF₃—, C₂F₅—, CH₃CF₂CF₂—, HCF₂CF₂CH₂—, CF₃CH₂—, HCF₂CF₂CF₂CF₂CH₂—, H (CF₂CF₂)₄CH₂—, CF₃CF₂CH₂—, (CF₃)₂CH—, (CF₃)₂CF—, C₈F₁₇CH₂CH₂—, 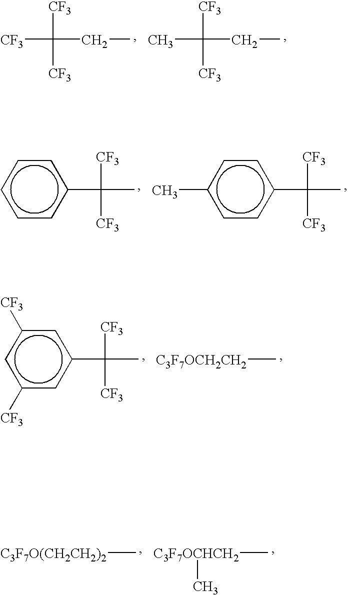
C₃F₇O—, CF₃O—, C₂F₅O—, CH₃CF₂CF₂O—, HCF₂CF₂CH₂O—, CF₃CH₂O—, HCF₂CF₂CF₂CF₂CH₂O—, H(CF₂CF₂)₄CH₂O—, C₂F₅CH₂—, (CF₃)₂CHO—, (CF₃)₂CFO—, C₈F₁₇CH₂CH₂—, 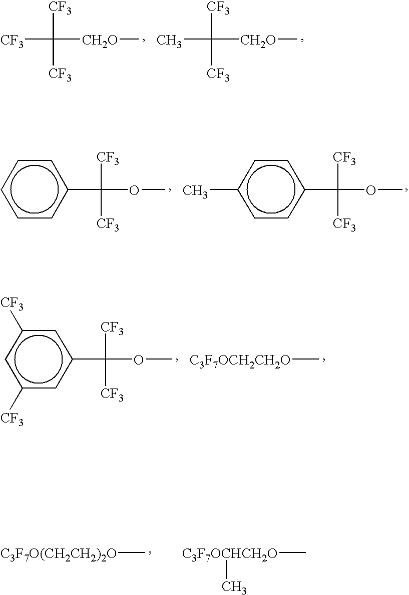
and the like, and particularly preferred are C₃F₇—, CF₃—, C₂F₅—, CF₃CH₂—, HCF₂CF₂CH₂—, CF₃CF₂CH₂—, (CF₃)₂CH—, C₃F₇O—, CF₃O—, C₂F₅O—, CF₃CH₂O—, HCF₂CF₂CH₂—, CF₃CF₂CH₂—, (CF₃)₂CHO— and the like.

Also other examples of Ra are (v) monovalent organic groups Rx′ having 2 to 20 carbon atoms which have an ethylenic double bond at an end thereof.

Such organic groups are preferred since in addition to the same effects as mentioned above by the moieties of -D- and Ry in the ionic liquid type functional material, it is possible to obtain the fluorine-containing polymer having the fluorine-containing ether unit -D-, functional group Y and aromatic ring structure in its side chain by polymerization using the ethylenic double bond, thereby making it possible to obtain the ionic liquid type polymer from this fluorine-containing polymer.

If the number of carbon atoms of the organic group constituting Rx′ is too high, a viscosity is increased and lowering of polymerizability is recognized. Therefore the number of carbon atoms is preferably from 1 to 10, more preferably from 1 to 5.

Concretely Rx′ is a moiety represented by the following formula:
CX¹X²═CX³—(CX⁴X⁵)ₙ₃(C═O)ₙ₄(O)ₙ₆—
wherein X¹, X², X⁴ and X⁵ are the same or different and each is hydrogen atom or fluorine atom; X³ is selected from hydrogen atom, fluorine atom, CH₃ and CF₃; n3, n4 and n6 are the same or different and each is 0 or 1, and more concretely there are radically polymerizable moieties such as vinyl group, vinyl ether group, allyl ether group, acryloyl group, methacryloyl group and α-fluoroacryloyl group.

Examples thereof are:
CH₂═CF—CF₂O—, CH₂═CH—CF₂O—, CF₂═CF—CF₂O—, CF₂═CF—CF₂CF₂O—, CF₂═CF—O—, CH₂═CH—O—, CF₂═CF—CH₂O—, CF₂═CF—CF₂CF₂CH₂—O—, 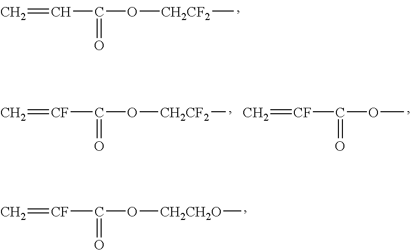
and the like.
(vi) Monovalent Organic Groups Ry′ Having 2 to 20 Carbon Atoms Which Have an Aromatic Ring Structure and at Least One Selected from the Basic Functional Groups Y¹ and/or the Salts Y² of the Basic Functional Groups

Concretely the same Ry as mentioned supra can be used preferably, thereby being capable of introducing not less than two Ry to one molecule of the compound of the formula (1) of the present invention and enabling functions of the ionic liquid type functional material to be exhibited more effectively.

Preferred examples of Ry′ are the same as the examples of Ry (-A-Ry″) raised supra.

The aromatic compound of the formula (1) to be used for the ionic liquid type functional material of the present invention is comprised of the fluorine-containing ether moiety -D- to be bonded to the moiety Ry of the aromatic ring structure containing the functional group Y and the residue Ra bonded to another end of -D-, in which to the moiety Ry is bonded at least one -D- (-D-Ra) or may be bonded 2 to 4-D- (-D-Ra) (m is from 2 to 4 in the formula (1)).

Preferred examples of the aromatic compounds of the formula (1) are those obtained in combination of the above-mentioned preferred examples of the fluorine-containing ether unit -D-, preferred examples of the moiety Ry having the functional group Y and aromatic ring structure, preferred examples of the bond for bonding D to Ry and preferred examples of the residue Ra.

Examples of the aromatic compound of the formula (1) when m is 1 are as follows.

(1-a) Aromatic Compounds in Which -D- and Ry are Bonded Directly and Ra is a Perfluoroalkyl Group

Preferred examples thereof are: 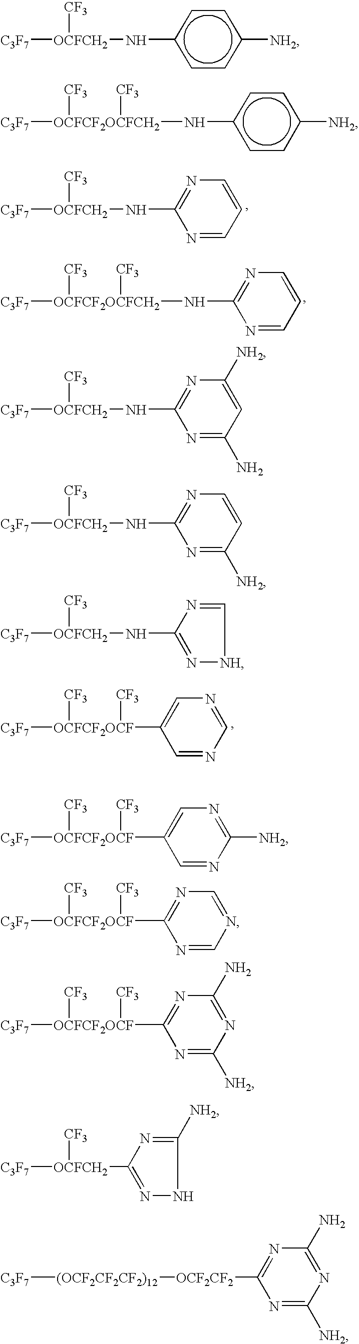 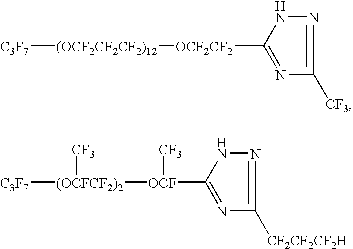
and the like.
(1-b) Aromatic Compounds in which -D- and Ry are Bonded via an Amide Bond and Ra is a Perfluoroalkyl Group

Preferred examples thereof are: 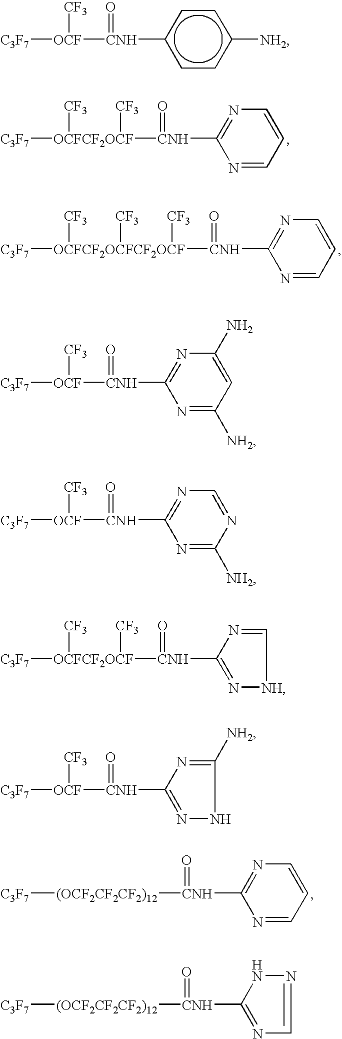
and the like.
(1-c) Aromatic Compounds in Which -D- and Ry are Bonded via an Amidine Bond and Ra is a Perfluoroalkyl Group

Preferred examples thereof are: 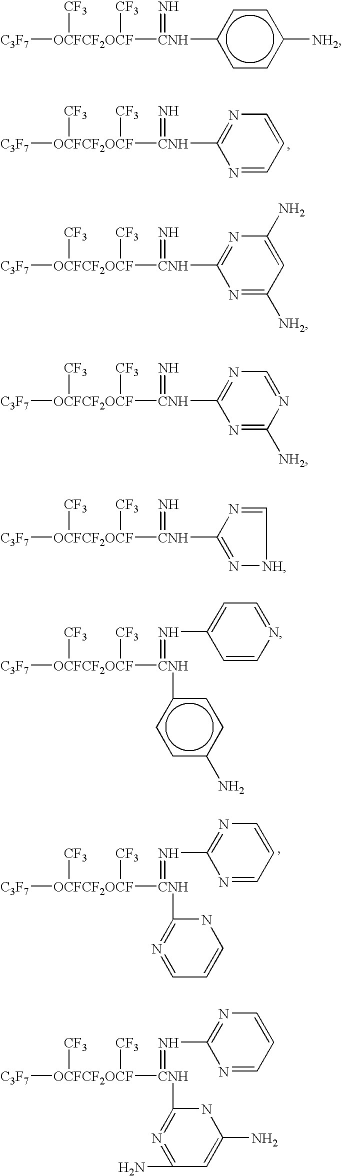
and the like.
(1-d) Aromatic Compounds of the Formula (1) When m is 2

Preferred examples thereof are: 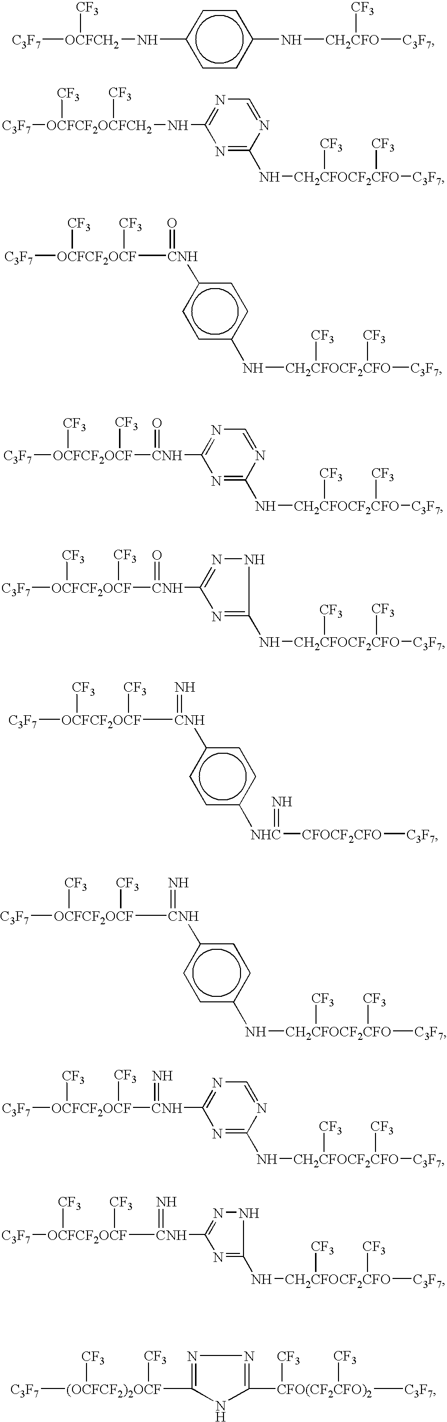 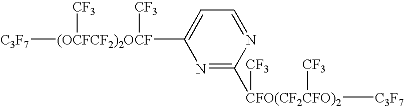
and the like.
(1-e) Aromatic Compounds in Which -D- and Ry are Bonded Directly and Ra has an Ethylenic Double Bond at its End

Preferred examples thereof are: 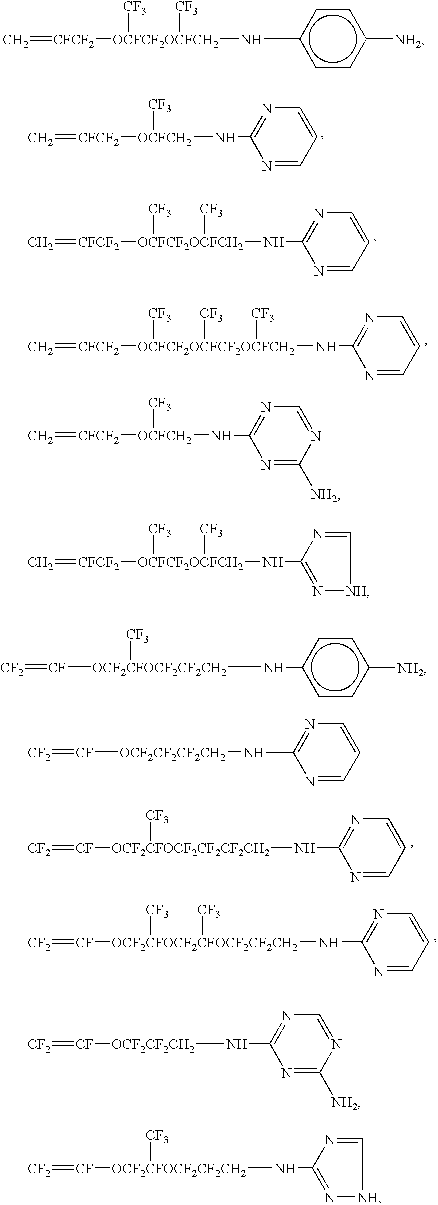 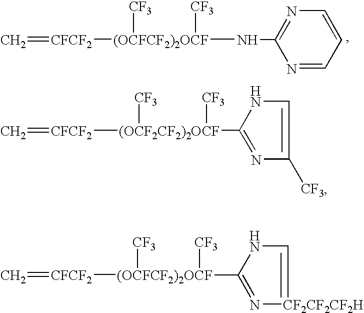
and the like.
(1-f) Aromatic Compounds in Which -D- and Ry are Bonded via an Amide Bond and Ra has an Ethylenic Double Bond at its End

Preferred examples thereof are: 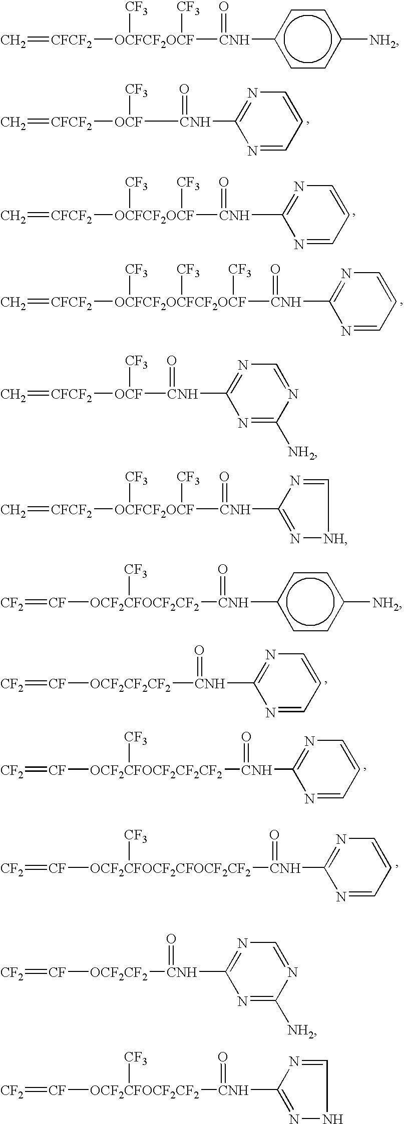
and the like.
(1-g) Aromatic Compounds in Which -D- and Ry are Bonded via an Amidine Bond and Ra has an Ethylenic Double Bond at its End

Preferred examples thereof are: 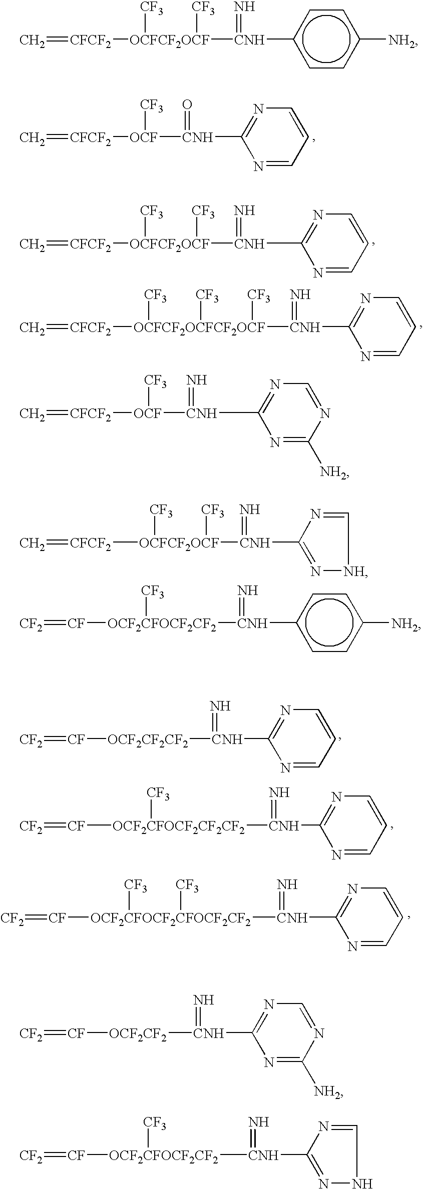
and the like.

The second of the ionic liquid type functional material of the present invention contains a polymer obtained by polymerizing a fluorine-containing ethylenic monomer which has, in the same side chain thereof, the fluoroether unit -D¹- and the moiety Ry¹ of aromatic ring structure having basic functional group and/or salt of the basic functional group.

Namely, the first of the polymer to be used for the second ionic liquid type functional material of the present invention is a fluorine-containing polymer represented by the formula (M-1):
-(M1)-(A1)- (M-1)
wherein the structural unit M1 is at least one selected from structural units derived from ethylenic monomers having, in a side chain thereof,
a moiety represented by the formula (2):
-D¹-Ry¹ (2)
in which -D¹- is a fluoroether unit represented by the formula (2-1): [symbol]
O—R¹ [symbol] ₙ₁ or [symbol] R¹—O [symbol] ₙ₁ (2-1)
wherein R¹ is at least one selected from divalent fluorine-containing alkylene groups having 1 to 5 carbon atoms in which at least one of hydrogen atoms is replaced by fluorine atom; n is an integer of from 1 to 20; Ry¹ is a monovalent organic group having 2 to 30 carbon atoms which has at least one selected from basic functional groups Y¹ and/or salts Y² of the basic functional groups and contains an aromatic ring structure, provided that a unit of —O—O— is not contained in the structural unit M1 and the formula (2-1); the structural unit A1 is a structural unit derived from a monomer being copolymerizable with the monomer being capable of providing the structural unit M1, and the structural units M1 and A1 are contained in amounts of from 1 to 100% by mole and from 0 to 99% by mole, respectively.

The second polymer of the present invention is a fluorine-containing polymer represented by the formula (M-2):
-(M2)-(A2)- (M-2)
wherein the structural unit M2 is a structural unit derived from an ethylenic monomer having, in a side chain thereof, a moiety represented by the formula (3):
-Ry² [symbol] D¹-Ra¹]ₘ₁ (3)
in which Ry² is a di-, tri- or tetra-valent organic group having 2 to 30 carbon atoms which has at least one of basic functional groups Y¹ and/or salts Y² of the basic functional groups and contains an aromatic ring structure; Ra¹ is a monovalent organic group which has 1 to 20 carbon atoms and does not contain D¹, and when m1 is not less than 2, two or more of Ra¹ may be the same or different; m1 is an integer of from 1 to 3; D¹ is selected from the same units as defined in the above-mentioned formula (2), and when m1 is not less than 2, two or more of D¹ may be the same or different, provided that a unit of —O—O— is not contained in the structural unit M2 and the formulae (2-1); the structural unit A2 is a structural unit derived from a monomer being copolymerizable with the monomer being capable of providing the structural unit M2, and the structural units M2 and A2 are contained in amounts of from 1 to 100% by mole and from 0 to 99% by mole, respectively.

Both of the fluorine-containing polymers of the formulae (M-1) and (M-2) to be used for the ionic liquid type functional material of the present invention are ethylenic polymers and are characterized by having, in one side chain thereof, the fluoroether unit -D- and the moiety Ry¹ of aromatic ring structure having the basic functional group Y¹ and/or the salt Y² of the basic functional group.

When Ry¹ is introduced, it is possible to effectively impart the mentioned desired functions of the ionic liquid type functional material by the basic functional group and polar aromatic structure contained therein without lowering oxidation resistance and heat resistance.

However only by the introduction of Ry¹ moiety to the polymer, reversely solidification or increase in a viscosity occurs, and functions of the ionic liquid are impaired.

The present inventors have found that when the ether unit having fluorine atom is introduced to the same side chain having Ry¹, a polymer which is usually solidified or undergoes increase in a viscosity is effectively liquefied or undergoes decrease in a viscosity.

Further the introduction of the fluoroether unit has good effect on oxidation resistance, heat resistance and chemical resistance.

The side chain structure of the fluorine-containing polymer (M-1) is, as shown in the formula (2), characterized by having Ry¹ at an end of its side chain via the fluoroether unit -D¹-, and is preferred since functions of the functional group contained in Ry¹ can be exhibited more effectively.

On the other hand, the side chain structure of the fluorine-containing polymer (M-2) is preferred since the fluoroether unit -D¹- and its residue Ra¹ are located at an end of its side chain via the moiety Ry² of aromatic ring structure having a functional group, thereby enabling the fluorine-containing polymer to be effectively liquefied or enabling the viscosity thereof to be effectively decreased.

In the fluorine-containing polymers (M-1) and (M-2) to be used for the ionic liquid type functional material of the present invention, the fluoroether unit -D¹- forming the side chain structure is selected from those represented by the formula (2-1). With respect to kind of the fluorine-containing alkylene group R¹, fluorine content, preferred examples and the number n1 of repeat units of fluoroether, there can be preferably similarly used the preferred -D- (R, n) of the formula (1-1) and examples thereof which are raised supra in the aromatic compound of the formula (1) to be used for the mentioned ionic liquid type functional material.

In the fluorine-containing polymers (M-1) and (M-2) to be used for the ionic liquid type functional material of the present invention, with respect to the moieties Ry¹ and Ry² of aromatic ring structure having the basic functional group Y¹ and/or the salt Y² of the basic functional group, concretely kinds of the basic functional group Y¹ and/or the salt Y² of the basic functional group contained in those moieties, kind and structure of the aromatic ring structure and further a method of bonding to the fluoroether unit -D¹- and kind of the bond, those which are raised supra in the aromatic compound of the formula (1) to be used for the mentioned ionic liquid type functional material can be similarly used preferably also for the fluorine-containing polymers of the present invention. Preferred examples thereof are those selected from the preferred examples of Ry raised supra.

Also in the moiety of the formula (3) constituting the side chain of the structural unit M2 of the polymer of the formula (M-2), Ra¹ is a residue bonded to the end of -D- and is not limited particularly as far as it is selected from monovalent organic groups having 1 to 20 carbon atoms. Preferred Ra and examples thereof which are explained in the aromatic compound of the formula (1) to be used for the ionic liquid type functional material can be preferably used similarly as Ra¹.

Particularly preferred are fluorine-containing alkyl groups (Rx represented by (iv) mentioned supra) which have 1 to 20 carbon atoms and may have ether bond since the polymer can be effectively liquefied and a viscosity thereof can be decreased effectively and further since oxidation resistance and heat resistance are excellent.

The same examples of Rx as in (iv) explained supra are preferred examples of Ra¹.

The structural unit M1 of the fluorine-containing polymer of the formula (M-1) is not limited particularly as far as a part or the whole of its side chain structure is a structural unit of an ethylenic monomer having the structure of the formula (2). Particularly preferred is a structural unit represented by the formula (2-2): 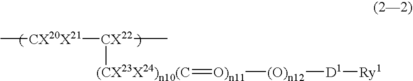
wherein X²⁰, X²¹, X²³ and X²⁴ are the same or different and each is hydrogen atom or fluorine atom; X²² is selected from hydrogen atom, fluorine atom, CH₃ and CF₃; n10 is 0 or an integer of 1 or 2; n11 and n12 are the same or different and each is 0 or 1; D¹ and Ry¹ are as defined in the formula (2), because various functions of the ionic liquid type functional material can be effectively exhibited and further oxidation resistance and heat resistance are excellent.

Preferred examples of the structural unit M1 of the formula (2-2) are: 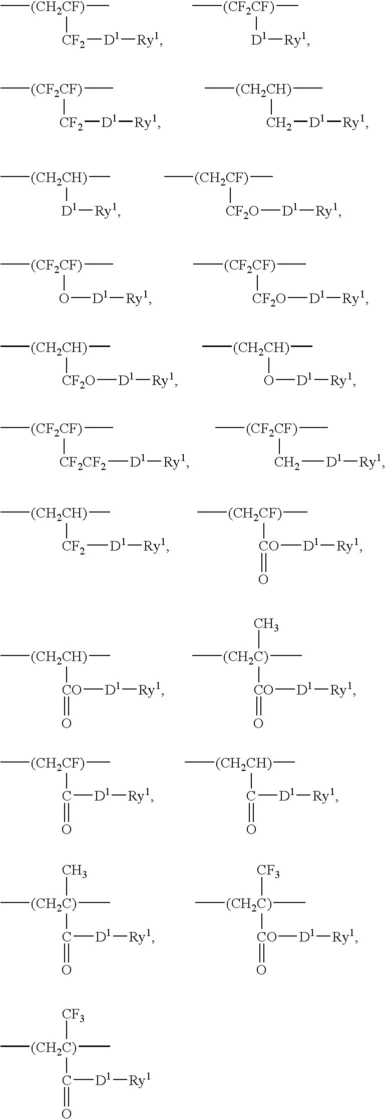
and the like, and particularly preferred structural unit of the formula (2-2) is a structural unit represented by the formula (2-3): 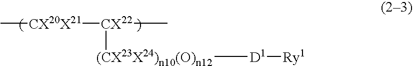
wherein X²⁰, X²¹, X²², X²³, X²⁴, n10 and n12 are as defined in the formula (2-2); D¹ and Ry¹ are as defined in the formula (2), particularly from the viewpoint of oxidation resistance, heat resistance and chemical resistance.

Preferred examples of the structural unit of the formula (2-3) are: 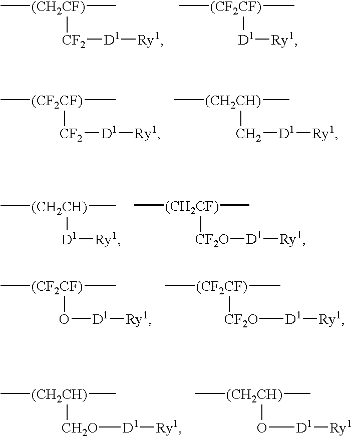
and the like, and particularly preferred structural units are: 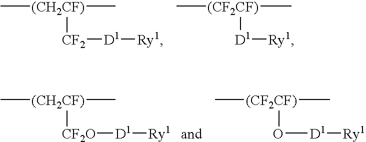
from the viewpoint of oxidation resistance, heat resistance and chemical resistance.

Examples of those preferred structural unit M1 are: 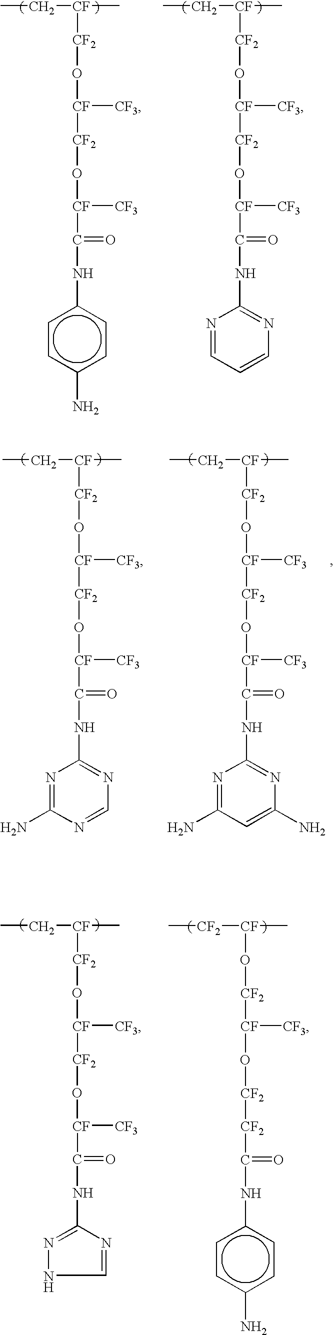 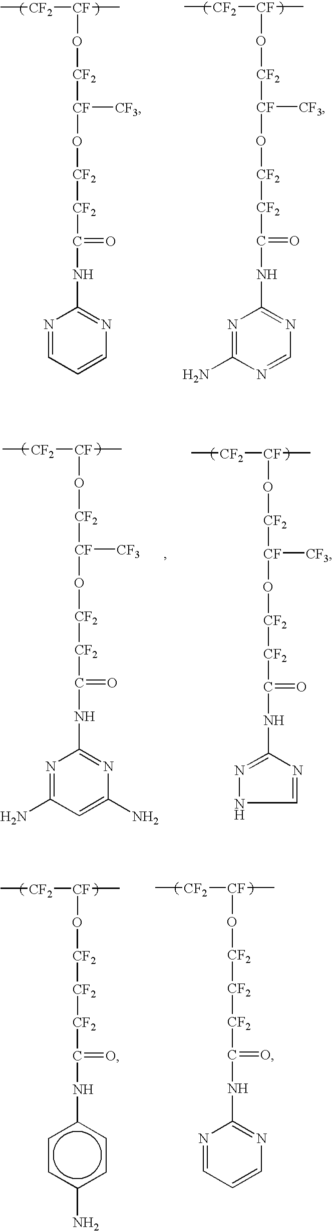 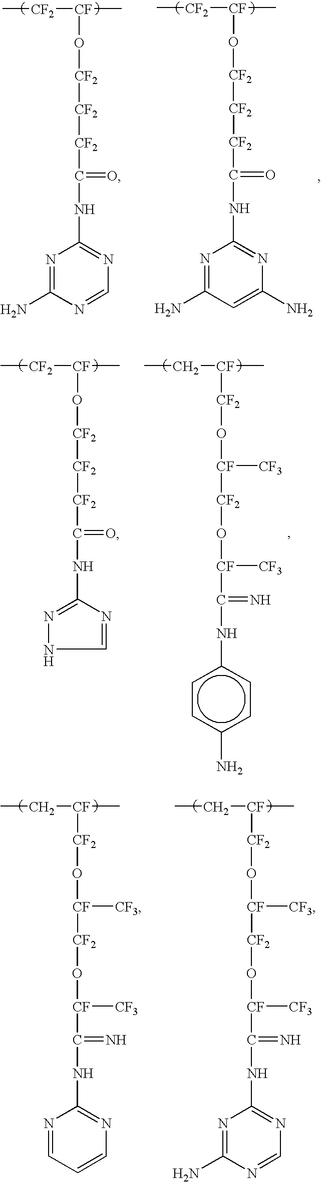 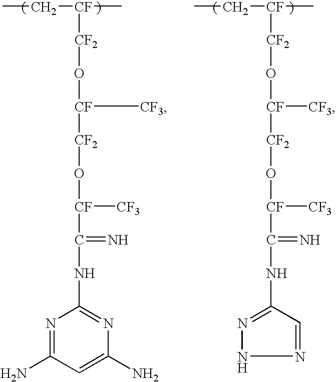
and the like.

The structural unit M2 of the fluorine-containing polymer of the formula (M-2) is not limited particularly as far as a part or the whole of its side chain structure is a structural unit of an ethylenic monomer having the structure of the formula (3). Particularly preferred is a structural unit represented by the formula (3-2): 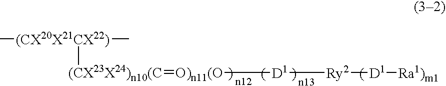
wherein X²⁰, X²¹, X²², X²³, X²⁴, n10, n11 and n12 are as defined in the formula (2-2); n13 is 0 or 1; D¹ is as defined in the formula (2), and when n13 is 1 and/or when m1 is not less than 2, two or more of D¹ may be the same or different; Ry², Ra¹ and m1 are as defined in the formula (3), because various functions of the ionic liquid type functional material can be effectively exhibited and further oxidation resistance and heat resistance are excellent.

More preferred is a structural unit represented by the formula (3-3): 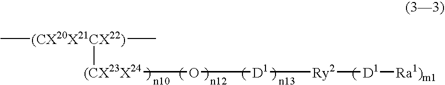
wherein X²⁰, X²¹, X²², X²³, X²⁴, n10, n12, n13 and D¹ are as defined in the formula (3-2); Ry², Ra¹ and m1 are as defined in the formula (3), from the viewpoint of excellent oxidation resistance and heat resistance like the structural unit M1 of the polymer of the formula (M-1).

Preferred examples of the structural unit M2 are the same as the examples of the structural unit M1 mentioned supra, in which the side chain portion:
-D¹-Ry¹
is replaced by the portion represented by: [symbol]
D¹ [symbol] ₙ₁₃Ry² [symbol] D¹-Ra¹]ₘ₁
wherein n13, D¹, Ry², Ra¹ and m1 are as defined in the formula (3-3).

Preferred examples of the structural unit M2 of the polymer of the formula (M-2) are: 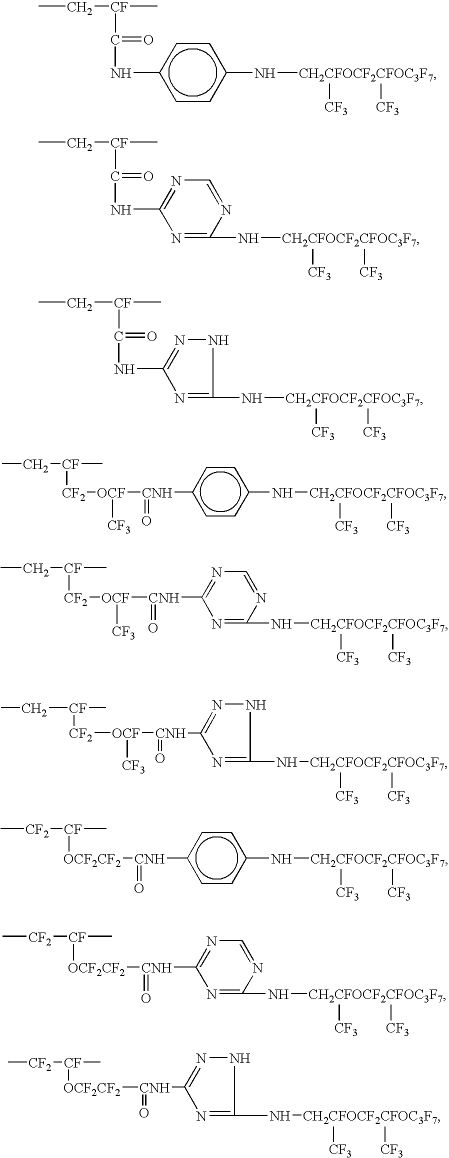
and the like.

In the present invention, the polymer of the formula (M-1) contains the structural unit M1 in an amount of not less than 1% by mole, and the polymer of the formula (M-2) contains the structural unit M2 in an amount of not less than 1% by mole. The polymers (M-1) and (M-2) may be copolymers containing the structural units A1 and A2, respectively derived from copolymerizable monomers.

The polymer may be a homopolymer of the structural unit M1 (or M2).

The structural unit A1 (or A2) is a structural unit derived from the monomer copolymerizable with the monomer being capable of providing the structural unit M1 (or M2) and is usually selected so that the fluorine-containing polymer becomes a non-crystalline polymer.

Thus the polymer can be liquefied and a viscosity can be decreased.

Also preferred as the structural unit A1 (or A2) are those which do not lower characteristics derived from the structural unit M1 (or M2), for example, oxidation resistance, heat resistance, liquidity and low viscosity.

From that point of view, it is preferable that the structural unit A1 (or A2) is a structural unit derived from a fluorine-containing ethylenic monomer.

Preferred as the structural unit derived from a fluorine-containing ethylenic monomer are those selected from structural units A1-1 (or A2-1) derived from fluorine-containing ethylenic monomers having 2 or 3 carbon atoms and at least one fluorine atom. Those structural units A1-1 (or A2-1) are preferred because oxidation resistance and heat resistance can be improved.

Concretely there are CF₂═CF₂, CF₂═CFC1, CH₂═CF₂, CFH═CH₂, CFH═CF₂, CF₂═CFCF₃, CH₂═CFCF₃, CH₂═CHCF₃ and the like. Among them, preferred are tetrafluoroethylene (CF₂═CF₂) and chlorotrifluoroethylene (CF₂═CFCl) because an effect of maintaining copolymerizability, oxidation resistance, heat resistance and chemical resistance is high.

The proportions of each structural unit in the fluorine-containing polymers of the formulae (M-1) and (M-2) are optionally selected depending on structures of the structural units M1 and M2, intended functions and applications. The structural units M1 (or M2) and A1 (or A2) are contained in amounts of preferably from 30 to 100% by mole and from 0 to 70% by mole, respectively, more preferably from 40 to 100% by mole and from 0 to 60% by mole, especially preferably from 60 to 100% by mole and from 0 to 40% by mole, further preferably from 70 to 100% by mole and from 0 to 30% by mole.

The number average molecular weight of the fluorine-containing polymers of the formulae (M-1) and (M-2) is from 500 to 1,000,000, preferably from 1,000 to 100,000, more preferably from 1,000 to 50,000, particularly preferably from 2,000 to 20,000.

If the molecular weight is too low, there is a case where there arises a problem that heat resistance is lowered or mechanical properties are lowered. Also a too high molecular weight is not preferred because there is a possibility of increasing a viscosity.

The aromatic compound of the formula (1) and the fluorine-containing polymers of the formulae (M-1) and (M-2) of the present invention have various properties and functions as mentioned supra and can be used alone or in a mixture with other components as the ionic liquid type functional material. Embodiments thereof are optionally selected depending on intended functions and applications.

Examples of the ionic liquid type functional material are, for instance, ionic liquid, electrolyte for solar cell, lubricant, acid-removing agent, actuator material and the like.

Other components which can be blended may be optionally selected depending on intended functional material. For example, in the case of using as a lubricant and actuator material, an organic acid or inorganic acid may be blended, and in the case of an electrolyte of solar cell, an organic solvent or inorganic acid may be blended. Also in the case of using as an ionic liquid, an other ionic liquid or organic solvent may be blended.

The above-mentioned organic acid and inorganic acid include both of BrØnsted acids releasing H⁺ and Lewis acids not releasing H⁺.

There may be used an optional acid as the above-mentioned acid. Examples of the BrØnsted acid releasing H⁺ are inorganic acids such as tetrafluoroboric acid, tungstic acid, chromic acid, hexafluorophosphoric acid, perchloric acid, hexafluoroarsenic acid, nitric acid, sulfuric acid, phosphoric acid, hydrofluoric acid, hydrochloric acid, hydrobromic acid, hydriodic acid and thiocyanic acid; organic acids such as trifluoromethanesulfonic acid, pentafluoroethanesulfonic acid, heptafluoropropylsulfonic acid, bis(trifluoromethanesulfonyl)-imide, acetic acid, trifluoroacetic acid, propionic acid, benzenesulfonic acid, toluenesulfonic acid, n-hexanesulfonic acid, n-octylsulfonic acid, cetylsulfonic acid, p-chlorobenzenesulfonic acid, phenolsulfonic acid, 4-nitrotoluene-2-sulfonic acid, 2-sulfobenzoic acid, nitrobenzenesulfonic acid, sulfosuccinic acid and sulfosebacic acid; and the like. Other examples are acrylic acid, methacrylic acid, styrene having carboxylic acid in its side chain, styrene having sulfonic acid in its side chain, solid polymer acids such as a perfluorosulfonic acid polymer having sulfonic acid in its side chain represented by NAFFION (trademark of DuPont), perfluorocarboxylic acid polymer having carboxylic acid in its side chain represented by FLEMION (trademark of Asahi Glass Co., Ltd.), perfluorophosphoric acid polymer having phosphoric acid in its side chain and perfluoroimide polymer having sulfonyl imide in its trunk chain or side chain.

Examples of the Lewis acid not releasing H⁺ are inorganic acids such as oxides or halides (fluorides, chlorides, bromides, iodides) of boron, aluminum, silica and transition metals, for example, molybdenum, tungsten, antimony, chromium, titanium, cobalt, iron, manganese, nickel, vanadium, tantalum, osmium, copper and zinc, and organic acids such as m- or p-nitrotoluene, nitrobenzene, p-nitrofluorobenzene, p-nitrochlorobenzene, 2,4-dinitrotoluene, 2,4-dinitrofluorobenzene, 2,4,6-trinitrotoluene and 2,4,6-trichlorobenzene.

Those acids may be blended to the aromatic compounds of the formula (1) and the fluorine-containing polymers of the formulae (M-1) and (M-2). In the case of the aromatic compounds of the formula (1), blending of the acid is advantageous for uses as functional materials for lubricant and ionic liquid, and in the case of the fluorine-containing polymers of the formulae (M-1) and (M-2), a viscosity of the polymer is decreased by blending of the acid, and the obtained polymer of a salt having a low viscosity is suitable as an ionic liquid polymer as well as an electrolyte for solar cell.

Also a composition containing a high molecular weight organic or inorganic acid in the form of solid and the compound or polymer of the present invention is a material suitable for an actuator.

The aromatic compound of the formula (1) or the fluorine-containing polymer of the formula (M-1) or (M-2) and an acid can be mixed at an optional ratio. It is preferable that a ratio of the number (Nb1) of basic functional groups Y¹ (or salts Y² thereof) in the aromatic compound or the polymer to the number (Na1) of acid groups in the organic acid or inorganic acid is from 0.01 to 100, further preferably from 0.1 to 10.

Examples of components other than the acid which can be blended are, for instance, organic solvents, ionic liquids other than the aromatic compounds and polymers of the present invention and the like. Examples of the desirable organic solvent are organic solvents having a high polarity, for instance, nitrites such as acetonitrile and benzonitrile; carbonates such as ethylene carbonate, propylene carbonate, dimethyl carbonate and diethyl carbonate; ethers such as tetrahydrofuran, triglyme and tetraglyme; and amides such as dimethylformamide, dimethylacetamide and dimethylsulfoamide. Examples of the ionic liquid other than the compounds of the present invention are ionic liquids composed of a cation of imidazole or pyridine derivative and an anion of Cl⁻, Br⁻, PF₆⁻, BF₄⁻, SbF₆⁻, CF₃SO₃⁻, ⁻N(SO₂CF₃)₂, ⁻C(SO₂CF₃)₂ or ⁻OCOCF₃.

Next, examples of applications of the ionic liquid type functional material of the present invention are explained below individually. The functional material of the present invention is not limited to such examples. Explanations are made with respect to the aromatic compounds (compounds of the present invention), but can also be applied to the fluorine-containing polymers of the formulae (M-1) and (M-2).

With respect to advantages in the case of the functional material being in the form of polymer, it is preferable that the ionic liquid is a polymer because when using, for example, for an electrochemical electrolyte (for example, electrolyte for Li secondary battery, electrolyte for capacitor, etc.), a reaction solvent and a solvent for separation or extraction, there are advantages that separability from a solvent, thermal stability and easy film formation can be obtained by making a molecular weight high. Also it is preferred in the form of a polymer, because by taking advantage of the above-mentioned functions as the ionic liquid, applications to functional membranes such as a gas separation membrane and permselective membrane become possible. Further when using for an electrolyte for solar cell, it is preferable that the ionic liquid is a polymer because sealing stability and moldability are enhanced.

(Ionic Liquid)

Currently among organic salts, attention is directed to salts being in the form of liquid at room temperature as an ionic liquid. Those salts are liquids having characteristics of a salt, namely having high polarity and having no vaporization pressure. Therefore those salts are expected to be applied to various fields such as an electrochemical electrolyte (for example, electrolyte for Li secondary battery, electrolyte for capacitor, etc.), a reaction solvent and a solvent for separation and extraction since it is considered that the salts bring about an innovative revolution to conventional organic solvents (IONIC LIQUID, CMC Shuppan, 2003).

Various derivatives having a base skeleton of imidazole or pyridine are investigated as cations of ionic liquids. This is because a base skeleton of a cation which can effectively decrease a melting point and a viscosity is limited to imidazole and pyridine derivatives, and kind of a selectable cation is restricted.

According to the present invention, by making the aromatic compound contain the fluorine-containing ether, especially even in polycyclic aromatic compounds, a melting point thereof can be decreased, and further a viscosity can also be reduced. Those compounds are suitable as a cation material for an ionic liquid because of a low melting point and low viscosity thereof. If the fluorine-containing ether chain is long, decrease in a melting point and viscosity is recognized, but a dielectric constant is also decreased and the compound becomes unsuitable as an ionic liquid material. Therefore when the compound is used as an ionic liquid, it is desirable that the number of fluorine-containing ether units is from 1 to 10, further desirably from 1 to 5. Also many of the compounds of the present invention having basic functional group are easily liquefied by mixing with an acid, and thus a composition containing an acid and the compound of the present invention can be an ionic liquid easily.

Such an ionic liquid is useful as an electrochemical electrolyte, reaction solvent and solvent for separation and extraction mentioned above.

(Electrolyte for Solar Cell)

As mentioned above, an electrolyte of solar cell can be raised as an application of the aromatic compound of the present invention. For an electrolyte of dye-sensitized solar cell, currently acetonitrile is used as a solvent. However acetonitrile has problems such as (i) lowering of sealing property due to thermal swelling and shrinking and (ii) oxidation due to direct excitation of TiO₂ to be used for an electrode. For that reason, studies have been made using an ionic liquid and gel electrolyte (JP2000-58891A; Leading-edge Technology of Electrolyte for Dye-sensitized Solar Cell, Chapter 28 (CMC Shuppan, 2001)), but sufficient performances (conductivity, life, etc.) have not yet been obtained.

The aromatic compound of the present invention can be used for an electrolyte of dye-sensitized solar cell. In an electrolyte of dye-sensitized solar cell, conduction of a salt of I⁻/I₃⁻ is necessary, and when I⁻ is used as a counter anion of the salt of the compound of the present invention, the material of the present invention works as an excellent electrolyte.

The aromatic compound of the present invention has advantages that thermal swelling and shrinkage are reduced because its boiling point is high, and a viscosity of the aromatic compound is decreased and oxidation resistance thereof is enhanced by making the aromatic compound contain the fluorine-containing ether.

In order to increase the conductivity of I⁻/I₃⁻, it is necessary to increase a salt concentration. From this point of view, preferred are salts derived from an aromatic ring structure having a higher concentration of basic functional groups. Therefore preferred are compounds containing polyfunctional basic group in one Ry, and concretely more preferred are the mentioned aromatic ring structures in which the atom forming the basic functional group constitutes the aromatic ring structure at the same time (examples of Ry of (ii) and (iii) explained supra).

(Lubricant)

Perfluoro polyether has been used for long as a lubricant. Since perfluoro polyether has no adhesion to a substrate, in order to enhance adhesion, there is employed a method of introducing a functional group such as hydroxyl or carboxyl, or converting to a salt of carboxylic acid. For example, with respect to a lubricant to be used as a magnetic recording medium, there are a report that an ester compound of perfluoro polyether having hydroxyl or carboxyl is used as a lubricant (JP5-194970A) and a report that an amine salt compound of perfluoro polyether having carboxylic acid group at an end or both ends thereof is used as a lubricant (JP5-143975A, JP2001-216625A). However acidity of carboxylic acid at an end of perfluoro polyether is higher than that of usual hydrocarbon carboxylic acid. Therefore in the case of ester, hydrolysis easily occurs and there is a problem with long-term stability. Also even in the case of using an amine salt compound of perfluoro polyether having carboxylic acid at an end thereof, since acidity of the carboxylic acid at an end of perfluoro polyether is strong, free carboxylic acid influences a substrate. Therefore an excessive amount of aromatic amine must be contained in the presence of the salt, and there is a problem with long-term stability due to dropping out and vaporization of the low molecular weight aromatic amine from a substrate.

When the aromatic compound of the present invention is used as a lubricant, the lubricant must be in the form of a composition containing the aromatic compound and an acid because the compound itself has basicity and has an adverse effect on a substrate. However since the fluorine-containing ether chain of the compound of the present invention has basicity, the compound can be used as a lubricant component in the form of a composition containing a weak acid having less effect on a substrate, and there are such effects that long-term stability is high and further adhesion to a substrate is excellent since the compound contains an aromatic ring structure.

Among the compounds of the present invention, those containing basic functional group is easily liquefied by mixing with a liquid acid, and is liquefied even if the liquid acid is not more than a neutral point. Thus the composition containing a liquid acid and the aromatic compound of the present invention can be a compound having a low viscosity and being suitable as an ionic liquid.

(Acid-removing Agent)

While importance is attracted to environmental problems, there arises a problem with disposal of waste water and waste solvent resulting from synthesis of chemical substances. Especially when waste water contains acid components such as an organic acid, phosphoric acid, and oxides or halides of osmium or tin having a great influence on environment, treatment of an acid is not considered to have been completed only by neutralization, and it is necessary to remove an acid itself from waste water. For removing an acid from waste water, there is a case where an anionic polymer is used as an acid-removing agent from the viewpoint of easy separation from water and easy recycling. However there are few kinds of anionic polymers having good separability from water and oxidation resistance thereof is not always good. Therefore the number of recyclings is limited. Further in a process for removing an acid from waste water and a process for removing an acid for recycling, efficiency is not always good because those processes are liquid-solid reaction. The aromatic compound itself of the present invention containing a fluorine-containing ether is a liquid, and therefore a process for removing an acid is liquid-liquid reaction and reaction efficiency is high. Also when the fluorine-containing ether chain is short, there is a case where the compound of the present invention is dissolved in an acidic aqueous solution, and thereby making it difficult to separate an acid. Therefore an aromatic compound having a long fluorine-containing ether chain is preferred. Further since the aromatic compound of the present invention has a high boiling point, there is no fear of evaporation and recycling is easy.

(Actuator)

An actuator is expected to be applied to an artificial muscle and micro robot, and is a material changing its form by external stimulation such as heat, light and electricity. Among actuators, a material in which a polymer electrolyte gel undergoes swelling and shrinkage in response to an electric signal is highly expected to be put into practical use because an electric signal can be easily controlled (Science and Industry, 72(4), pp. 162 to 167 (1998)).

It is preferable that such an actuator shows a larger change in response to an electric potential because an intended deformation is made by a smaller electric potential. Currently solid polymer acid materials such as perfluorosulfonic acid membrane and perfluorocarboxylic acid membrane subjected to swelling with water are used as an actuator material, and studies are made to increase a degree of deformation in response to an electric potential.

In the present invention, for example, when the compound of the present invention having basic functional group or the polymer of the present invention is added to a solid polymer acid such as perfluorosulfonic acid membrane or perfluorocarboxylic acid membrane subjected to swelling with water, there is an effect of increasing an amount of change in its form as compared with the case of not adding the compound or polymer.

The functional material of the present invention, as mentioned above, can be used or mixed in the form of liquid or in the form of solid. Further the functional material can be formed into a solid membrane.

For forming the functional material into a solid membrane, known optional methods, for example, casting, impregnation and heat press are used.

In the case of forming the solid membrane, combinations of the acid and the compound or polymer are

(i) impregnation of the solid acid with the liquid aromatic compound having fluorine-containing ether according to the present invention,

(ii) impregnation of the liquid acid with the solid aromatic polymer having fluorine-containing ether according to the present invention,

(iii) composite of the solid aromatic polymer having fluorine-containing ether according to the present invention and the solid acid,

(iv) impregnation of an other solid membrane with a composition containing an acid and the compound or polymer, and the like.

The second of the present invention is a novel aromatic compound having a fluorine-containing ether chain.

The first of the novel aromatic compound having a fluorine-containing ether chain is included in the aromatic compounds to be used in the ionic liquid type functional material explained supra, and is one of the most preferred ionic liquid type functional materials. This compound is a novel compound which is not disclosed in patent publications and bulletins.

The first of the novel aromatic compound of the present invention is the aromatic compound which has a fluorine-containing ether chain and is represented by the formula (4):
[RX²-D² [symbol] ₘ₂Ry³ (4)
wherein -D²- is a fluoroether unit represented by the formula (4-1): [symbol]
O—R² [symbol] ₙ₂ or [symbol] R²—O [symbol] ₙ₂ (4-1)
in which R² is at least one selected from divalent fluorine-containing alkylene groups having 1 to 5 carbon atoms in which at least one of hydrogen atoms is replaced by fluorine atom; n2 is an integer of from 1 to 20, and when m2 is not less than 2, two or more of D² may be the same or different; Ry³ is a mono-, di-, tri- or tetra-valent organic group having 2 to 30 carbon atoms which has at least one of amines and/or salts of amines and contains an aromatic ring structure; Rx² is a fluorine-containing alkyl group which has 1 to 20 carbon atoms and may have ether bond, and when m2 is not less than 2, two or more of Rx² may be the same or different; m2 is an integer of from 1 to 4, provided that a unit of —O—O— is not contained in the above-mentioned formulae (4) and (4-1).

In the aromatic compound of the formula (4), the fluoroether unit -D²- is preferably the same as -D- in the aromatic compound (of the formula (1)) to be used in the ionic liquid type functional material explained supra. Also preferred examples of the fluoroether unit -D²- are the same as those of -D- explained supra.

In the aromatic compound of the formula (4), Ry³ is a mono-, di-, tri- or tetra-valent organic group having 2 to 30 carbon atoms which has at least one of amines and/or salts of amines and contains an aromatic ring structure. Concretely preferred examples of Ry³ are the same as those of Ry in the aromatic compound of the formula (1) explained supra except that the functional group is selected from amines and salts thereof.

Also in the aromatic compound of the formula (4), Rx² is a fluorine-containing alkyl group having 1 to 20 carbon atoms. Concretely Rx² is the same as the fluorine-containing alkyl group Rx which may have ether bond and is raised as a preferred group of Ra in the aromatic compound of the formula (1). Also preferred examples of the Rx² are the same as those of Rx exemplified supra.

Also preferred examples of the structure of the whole aromatic compound of the formula (4) are the same as those of (1-a), (1-b), (1-c) and (1-e) among the classified examples of the aromatic compound of the formula (1) explained supra.

Those exemplified aromatic compounds are novel compounds which are not disclosed in patent publications and bulletins, and are preferred compounds which can be used for applications such as an epoxy curing agent, water storage agent and surface-protecting agent in addition to the ionic liquid type functional material application.

The second of the novel aromatic compound of the present invention is the aromatic compound having a polymerizable ethylenic double bond among the aromatic compounds of the formula (1) to be used in the ionic liquid type functional material explained supra, and is preferred since the compound can provide a polymer by polymerization thereof, and the obtained polymer has, as the ionic liquid type polymer, excellent performance explained supra in addition to the functions of the ionic liquid.

The second of the novel aromatic compound of the present invention is the aromatic compound which has a fluorine-containing ether chain and is represented by the formula (5):
CX¹X²═CX³—(CX⁴X⁵)ₙ₃(C═O)ₙ₄-D²-Ry⁴ (5)
wherein X¹, X², X⁴ and X⁵ are the same or different and each is hydrogen atom or fluorine atom; X³ is selected from hydrogen atom, fluorine atom, CH₃ and CF₃; n3 and n4 are the same or different and each is 0 or 1; Ry⁴ is a monovalent organic group having 2 to 30 carbon atoms which has at least one of amines and/or salts of amines and contains an aromatic ring structure; D² is as defined in the formula (4).

In the aromatic compound of the formula (5), preferred examples of -D²- are the same as those of -D²- in the aromatic compound of the formula (4). -Ry⁴ is preferably the same as the monovalent organic group among -Ry³ in the aromatic compounds of the formula (4) which is a mono-, di-, tri- or tetra-valent organic group having 2 to 30 carbon atoms which has at least one of amines and/or salts of amines and contains an aromatic ring structure.

Examples of the aromatic compounds of the formula (5) are: 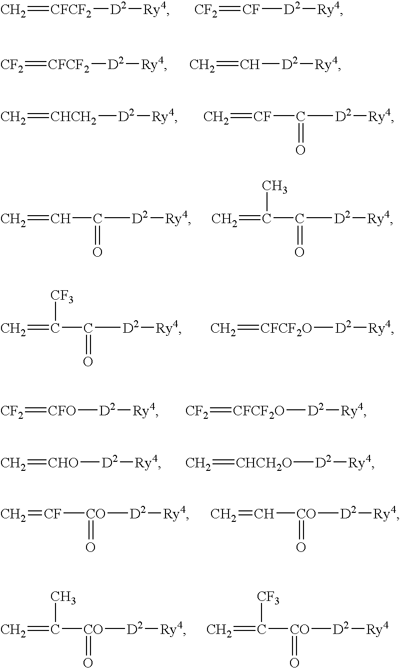
and the like, and particularly preferred are aromatic compounds which has a fluorine-containing ether chain and is represented by the formula (6):
CX¹X²═CX³—(CX⁴X⁵)ₙ₃-D²-Ry⁴ (6)
wherein X¹, X², X³, X⁴, X⁵, n3, D² and Ry⁴ are as defined in the formula (5), from the viewpoint of excellent homopolymerizability and copolymerizability with a fluorine-containing ethylenic monomer and also from the point that oxidation resistance and heat resistance can be imparted effectively to the obtained polymer.

Examples of the aromatic compounds of the formula (6) are:

CH₂═CFCF₂-D²-Ry⁴, CF₂═CF-D²-Ry⁴,

CF₂═CFCF₂-D²-Ry⁴, CH₂═CH-D²-Ry⁴,

CH₂═CHCH₂-D²-Ry⁴,

CH₂═CFCF₂O-D²-Ry⁴, CF₂═CFO-D²-Ry⁴,

CF₂═CFCF₂O-D²-Ry⁴, CH₂═CHO-D²-Ry⁴,

CH₂═CHCH₂O-D²-Ry⁴

and the like, and particularly preferred are:

CH₂═CFCF₂-D²-Ry⁴, CF₂═CF-D²-Ry⁴,

CH₂═CFCF₂O-D²-Ry⁴ and CF₂═CFO-D²-Ry⁴

because polymerizability is high and oxidation resistance and heat resistance can be effectively imparted to the polymer.

Also preferred examples of the structure of the whole aromatic compound of the formula (6) are the same as those of (1-e), (1-f) and (1-g) among the classified examples of the aromatic compound of the formula (1).

The third of the present invention relates to a novel fluorine-containing polymer which is included in the polymers of the formula (M-1) to be used in the ionic liquid type functional material, and is one of the most preferred ionic liquid type functional materials.

The novel polymer of the present invention is the fluorine-containing polymer which has a number average molecular weight of from 500 to 1,000,000 and is represented by the formula (M-3):
-(M3)-(A3)- (M-3)
wherein the structural unit M3 is a structural unit represented by the formula (7): 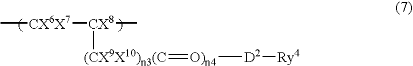
wherein X⁶, X⁷, X⁹ and X¹⁰ are the same or different and each is hydrogen atom or fluorine atom; X⁸ is selected from hydrogen atom, fluorine atom, CH₃ and CF₃; n3 and n4 are the same or different and each is 0 or 1; D² and Ry⁴ are as defined in the formula (5); the structural unit A3 is a structural unit derived from a monomer being copolymerizable with the monomer being capable of providing the structural unit M3, and the structural units M3 and A3 are contained in amounts of from 1 to 100% by mole and from 0 to 99% by mole, respectively.

The polymer of the formula (M-3) is a novel polymer which is not disclosed in patent publications and bulletins.

The polymer of the formula (M-3) of the present invention is obtained by polymerizing the aromatic compound of the formula (5) having an ethylenic double bond, and may be a homopolymer containing only the structural unit M3 or may be a copolymer of the monomer of the formula (5) being capable of providing M3 and a copolymerizable monomer.

Examples of the structural unit M3 in the polymer of the formula (M-3) are: 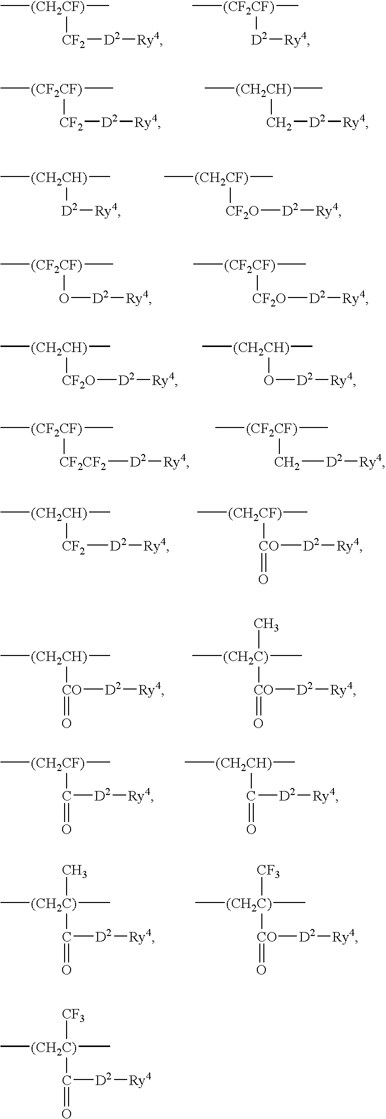
and the like, and particularly preferred as the structural unit M3 are structural units represented by the formula (8): 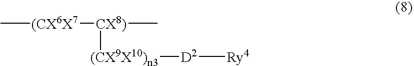
wherein X⁶, X⁷, X⁸, X⁹, X¹⁰, n3, D² and Ry⁴ are as defined in the formula (7), from the viewpoint of oxidation resistance, heat resistance and chemical resistance.

Preferred examples of the structural units of the formula (8) are: 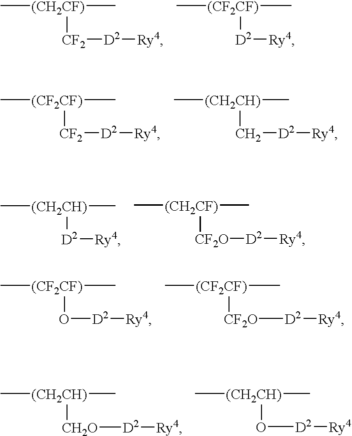
and the like, and particularly preferred are structural units such as: 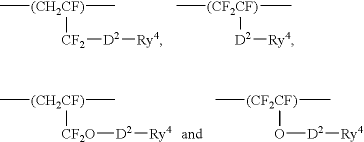
from the viewpoint of oxidation resistance, heat resistance and chemical resistance.

Preferred examples of the whole structure of the preferred structural unit M3 are the same as the examples of the structural unit M1 raised in the formula (M-1) to be used in the ionic liquid type functional material explained supra.

The polymer (M-3) of the present invention contains the structural unit M3 in an amount of not less than 1% by mole and may be a copolymer containing the structural unit A3 derived from a copolymerizable monomer. The polymer (M-3) may be a homopolymer of the structural unit M3.

Preferred as the structural unit A3 are those which do not lower the characteristics derived from the structural unit M3, for example, oxidation resistance, heat resistance, liquidity and low viscosity.

From that point of view, it is preferable that the structural unit A3 is a structural unit derived from a fluorine-containing ethylenic monomer.

Preferred examples of the structural unit derived from a fluorine-containing ethylenic monomer are those selected from the structural units A3-1 derived from fluorine-containing ethylenic monomers which have 2 or 3 carbon atoms and at least one fluorine atom.

The structural units A3-1 are preferred since oxidation resistance and heat resistance can be improved.

Concretely there are CF₂═CF₂, CF₂═CFCl, CH₂═CF₂, CFH═CH₂, CFH═CF₂, CF₂═CFCF₃, CH₂═CFCF₃, CH₂═CHCF₃ and the like, and among them, preferred are tetrafluoroethylene (CF₂═CF₂) and chlorotrifluoroethylene (CF₂═CFCl) because an effect of maintaining copolymerizability, oxidation resistance, heat resistance and chemical resistance is high.

The proportions of each structural unit in the fluorine-containing polymers of the formula (M-3) are optionally selected depending on the structure of the structural unit M3, intended functions and applications. The structural units M3 and A3 are contained in amounts of preferably from 30 to 100% by mole and from 0 to 70% by mole, respectively, more preferably from 40 to 100% by mole and from 0 to 60% by mole, especially preferably from 60 to 100% by mole and from 0 to 40% by mole, further preferably from 70 to 100% by mole and from 0 to 30% by mole.

The number average molecular weight of the fluorine-containing polymer of the formula (M-3) is from 500 to 1,000,000, preferably from 1,000 to 100,000, more preferably from 1,000 to 50,000, particularly preferably from 2,000 to 20,000.

If the molecular weight is too low, there is a case where there arises a problem that heat resistance and mechanical properties are lowered. Also a too high molecular weight is not preferred because there is a possibility of increasing a viscosity.

The fluorine-containing polymer of the formula (M-3) of the present invention is most preferred as the ionic liquid type functional material explained supra, and in addition, is a preferred compound which can be used for applications such as an epoxy curing agent, water storage agent and surface-protecting agent.

### EXAMPLE

The present invention is then explained below by means of examples and preparation examples, but is not limited to them.

In the following Examples, equipment and measuring conditions used for evaluation of physical properties are as follows.

(1) NMR: AC-300 available from BRUKER CO., LTD.

Measuring conditions of ¹H-NMR: 300 MHz (tetramethylsilane=0 ppm)

Measuring conditions of ¹⁹F-NMR: 282 MHz (trichlorofluoromethane=0 ppm)

(2) IR analysis: Measuring is carried out at room temperature with a Fourier-transform infrared spectrophotometer 1760X available from Perkin Elmer Co., Ltd.

(3) GPC: The number average molecular weight is calculated from the data measured by gel permeation chromatography (GPC) by using GPC HLC-8020 available from Toso Kabushiki Kaisha and columns available from Shodex (one GPC KF-801, one GPC KF-802 and two GPC KF-806M are connected in series) and flowing tetrahydrofuran (THF) as a solvent at a flowing rate of 1 ml/min.

(4) TGA measurement: A 10% thermal decomposition temperature (T_{d10}) and 50% thermal decomposition temperature (T_{d50}) are calculated from the data obtained when heating up from room temperature at a rate of 10° C./min by using TG/DTA-6200 available from Seiko Instruments Kabushiki Kaisha.

### Example 1

### Synthesis of perfluoro(2,5-bistrifluoromethyl-3,6-dioxanonanoic acid)-(2-pyrimidine)amide

Into a 500 ml four-necked flask equipped with a thermometer and dropping funnel were poured 150 ml of dehydrated DMF, 57 g of aminopyrimidine and 42 g of triethylamine in a nitrogen atmosphere. On an ice bath, 176 g of perfluoro(2,5-bistrifluoromethyl-3,6-dioxanonanoic acid chloride: 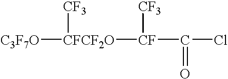
was slowly added dropwise with stirring. After completion of the addition, the mixture temperature was increased to room temperature gradually and stirring was carried out at room temperature for one hour. The reaction solution was subjected to separation with an acid and water to take out an oil layer. After drying of the oil layer with magnesium sulfate, distillation was carried out under reduced pressure and 142 g of perfluoro(2,5-bistrifluoromethyl-3,6-dioxanonanoic acid)-(2-pyrimidine)amide: 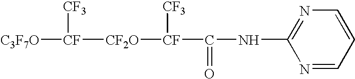
was obtained. According to ¹⁹F-NMR and ¹H-NMR analyses, it was confirmed that the obtained product was the above-mentioned compound. This compound was soluble in acetone and ethyl acetate and was in the form of liquid at room temperature. Also as a result of TGA measurement in the air, Tdlo was 159° C. and T_{d50} was 188° C.

¹⁹F-NMR (CD₃COCD₃): −78 to −80 ppm (7F), −82 to −85 ppm (4F), −92 ppm (2F), −115 ppm (2F), −132 ppm (1F), −145.0 ppm (1F) ¹H-NMR (CD3COCD3): 7.2 ppm (1H), 8.7 ppm (2H), 9.0 ppm (1H)

### Example 2

### Synthesis of N-perfluoro(1,1-dihydro-2,5-bistrifluoromethyl-3,6-dioxanonanoyl) -N-(2-pyrimidine) Into a 500 ml four-necked flask equipped with a thermometer and dropping funnel were poured 150 ml of sulfolane, 60 g of perfluoro(1,1-dihydro-2,5-bistrifluoromethyl-3,6-dioxanonanoyl)-ortho-nitrobenzenesulfonate:

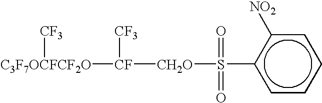 and 34 g of aminopyrimidine in a nitrogen atmosphere, followed by stirring at 170° C. for 24 hours in a nitrogen atmosphere. The reaction solution was poured into water, and a solid was removed by filtration. The filtrate was subjected to separation with water/ethyl acetate to take out an oil layer. After drying of the oil layer with magnesium sulfate, distillation was carried out under reduced pressure and 17 g of N-perfluoro(1,1-dihydro-2,5-bistrifluoromethyl-3,6-dioxanonanoyl)-N-(2-pyrimidine): 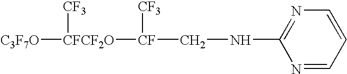
was obtained. According to ¹⁹F-NMR and ¹H-NMR analyses, it was confirmed that the obtained product was the above-mentioned compound. This compound was soluble in acetone and ethyl acetate and was in the form of liquid at room temperature. Also as a result of TGA measurement in the air, T_{d10} was 122° C. and T_{d50} was 138° C.

¹⁹F-NMR (CD₃COCD₃): −78 to −80 ppm (7F), −82 to −85 ppm (4F), −94 ppm (2F), −112 ppm (2F), −143 ppm (1F), −168 ppm (1F) ¹H-NMR (CD₃COCD₃): 2.8 ppm (1H), 3.6 ppm (2H), 7.2 ppm (1H), 8.7 ppm (2H), 9.0 ppm (1H)

### Example 3

### Synthesis of perfluoro(2,5,8-tristrifluoromethyl-3,6,9-trioxadodecanoic acid)-(2-pyrimidine)amide

Into a 500 ml four-necked flask equipped with a thermometer and dropping funnel were poured 150 ml of dehydrated DMF, 50 g of aminopyrimidine and 42 g of triethylamine in a nitrogen atmosphere. On an ice bath, 160 g of perfluoro(2,5,8-tristrifluoromethyl-3,6,9-trioxadodecanoic acid chloride): 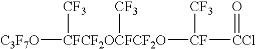
was slowly added dropwise with stirring. After completion of the addition, the mixture temperature was increased to room temperature gradually and stirring was carried out at room temperature for one hour. The reaction solution was subjected to separation with an acid and water to take out an oil layer. After concentration of the oil layer, 136 g of perfluoro(2,5,8-tristrifluoromethyl-3,6,9-trioxadodecanoic acid)-(2-pyrimidine)amide: 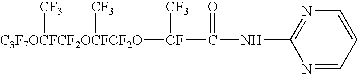
was obtained. According to ¹⁹F-NMR and ¹H-NMR analyses, it was confirmed that the obtained product was the above-mentioned compound. This compound was soluble in acetone and ethyl acetate and was in the form of liquid at room temperature. Also as a result of TGA measurement in the air, T_{d10} was 180° C. and T_{d50} was 209° C.

¹⁹F-NMR (CD₃COCD₃): −78 to −80 ppm (11F), −82 to −85 ppm (5F), −97 ppm (2F), −118 ppm (2F), −132 ppm (1F), −147 ppm (2F) ¹H-NMR (CD₃COCD₃): 7.2 ppm (1H), 8.7 ppm (2H), 9.0 ppm (1H)

### Example 4

### Synthesis of perfluoro(9,9-dihydro-2,5-bistrifluoromethyl-3,6-dioxanonenoic acid)-(4-aminophenyl)amide

Into a 500 ml four-necked flask equipped with a thermometer and dropping funnel were poured 120 ml of dehydrated DMF, 38.9 g of p-phenylenediamine and 18.3 g of triethylamine in a nitrogen atmosphere. Then 50.8 g of perfluoro(9,9-dihydro-2,5-bistrifluoromethyl-3,6-dioxanonenoic acid chloride) was slowly added dropwise at room temperature with stirring. After completion of the addition, stirring was carried out overnight at room temperature. To the reaction solution were added water and HCFCl41b for separation of solution to take out an oil layer. After drying of the oil layer with magnesium sulfate, heating was carried out under reduced pressure to remove the unreacted starting materials, etc. and 36.5 g of perfluoro(9,9-dihydro-2,5-bistrifluoromethyl-3,6-dioxanonenoic acid)-(4-aminophenyl)amide: 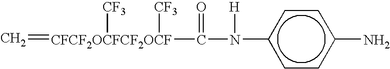
was obtained. According to ¹⁹F-NMR and ¹H-NMR analyses, it was confirmed that the obtained product was the above-mentioned compound. This compound was soluble in acetone and ethyl acetate and was in the form of solid at room temperature. Also as a result of TGA measurement in the air, T_{d10} was 213° C. and T_{d50} was 258° C.

¹⁹F-NMR (CD₃COCD₃): −73 ppm (2F), −78 to −80 ppm (4F), −82 to −85 ppm (4F), −124 ppm (1F), −132 ppm (1F), −145.0 ppm (1F) ¹H-NMR (CDCl₃): 5.1 ppm (1H), 5.3 ppm (1H), 6.7 ppm (2H), 7.2 ppm (2H), 7.6 ppm (2H), 8.1 ppm (1H)

### Example 5

### Synthesis of perfluoro(9,9-dihydro-2,5-bistrifluoromethyl-3,6-dioxanonenoic acid)-(2-aminopyrimidine)amide

Into a 500 ml four-necked flask equipped with a thermometer and dropping funnel were poured 80 ml of dehydrated THF, 14.8 g of 2-aminopyrimidine and 16.8 g of triethylamine in a nitrogen atmosphere. Then 52.0 g of perfluoro(9,9-dihydro-2,5-bistrifluoromethyl-3,6-dioxanonenoic acid chloride) was slowly added dropwise at room temperature with stirring. After completion of the addition, stirring was carried out overnight at room temperature. To the reaction solution were added water and HCFCl41b for separation of solution to take out an oil layer. After drying of the oil layer with magnesium sulfate, heating was carried out under reduced pressure to remove the unreacted starting materials, etc. and 46.7 g of perfluoro(9,9-dihydro-2,5-bistrifluoromethyl-3,6-dioxanonenoic acid)-(2-aminopyrimidine)amide: 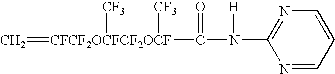
was obtained. According to ¹⁹F-NMR and ¹H-NMR analyses, it was confirmed that the obtained product was the above-mentioned compound. This compound was soluble in acetone and ethyl acetate and was in the form of liquid at room temperature. Also as a result of TGA measurement in the air, T_{d10} was 167° C. and T_{d50} was 198° C.

¹⁹F-NMR (CD₃COCD₃): −73 ppm (2F), −78 to −80 ppm (4F), −87 to −90 ppm (4F), −124 ppm (1F), −132 ppm (1F), −145.0 ppm (1F) ¹H-NMR (CDCl₃): 5.2 ppm (1H), 5.3 ppm (1H), 7.2 ppm (2H), 8.7 ppm (2H), 9.1 ppm (1H)

### Example 6

### Synthesis of perfluoro(9,9-dihydro-2,5-bistrifluoromethyl-3,6-dioxanonenoic acid)-(3-(1,2,4-triazole))amide

Into a 500 ml four-necked flask equipped with a thermometer and dropping funnel were poured 90 ml of dehydrated THF, 15.1 g of 3-amino-1,2,4-triazole and 24.1 g of triethylamine in a nitrogen atmosphere. On a water bath, 65.4 g of perfluoro(9,9-dihydro-2,5-bistrifluoromethyl-3,6-dioxanonenoic acid chloride) was slowly added dropwise with stirring. After completion of the addition, stirring was carried out overnight at room temperature. To the reaction solution were added water and HCFCl41b for separation of solution to take out an oil layer. After drying of the oil layer with magnesium sulfate, heating was carried out under reduced pressure to remove the unreacted starting materials, etc. and 67.0 g of perfluoro(9,9-dihydro-2,5-bistrifluoromethyl-3,6-dioxanonenoic acid)-(3-(1,2,4-triazole))amide: 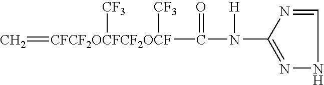
was obtained. According to ¹⁹F-NMR and ¹H-NMR analyses, it was confirmed that the obtained product was the above-mentioned compound. This compound was soluble in acetone and ethyl acetate and was in the form of solid at room temperature. Also as a result of TGA measurement in the air, T_{d10} was 188° C. and T_{d50} was 232° C.

¹⁹F-NMR (CD₃COCD₃): −73 ppm (2F), −78 to −80 ppm (4F), −87 to −90 ppm (4F), −124 ppm (1F), −132 ppm (1F), −145.0 ppm (1F) ¹H-NMR (CD₃COCD₃): 5.4 ppm (1H), 5.6 ppm (1H), 8.3 ppm (1H), 8.5 ppm (1H), 8.7 ppm (1H)

### Example 7

### Synthesis of perfluoro(12,12-dihydro-2,5,8-tristrifluoromethyl-3,6,9-trioxadodecenoic acid)-(2-aminopyrimidine)amide

Into a 500 ml four-necked flask equipped with a thermometer and dropping funnel were poured 100 ml of dehydrated THF, 9.89 g of 2-aminopyrimidine and 12.0 g of triethylamine in a nitrogen atmosphere. Thereto was slowly added dropwise 48.4 g of perfluoro(12,12-dihydro-2,5,8-tristrifluoromethyl-3,6,9-trioxadodecenoic acid chloride) at room temperature with stirring. After completion of the addition, stirring was carried out overnight at room temperature. To the reaction solution were added water and HCFCl41b for separation of solution to take out an oil layer. After drying of the oil layer with magnesium sulfate, heating was carried out under reduced pressure to remove the unreacted starting materials, etc. and 20.9 g of perfluoro(12,12-dihydro-2,5,8-tristrifluoromethyl-3,6,9-trioxadodecenoic acid)-(2-aminopyrimidine)amide: 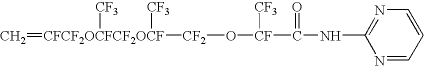
was obtained. According to ¹⁹F-NMR, ¹H-NMR and IR analyses, it was confirmed that the obtained product was the above-mentioned compound. This compound was a liquid. Also as a result of TGA measurement in the air, T_{d10} was 182° C. and T_{d50} was 254° C.

¹⁹F-NMR (CD₃COCD₃): −73 ppm (2F), −78 to −80 ppm (8F), −87 to −90 ppm (5F), −124 ppm (1F), −132 ppm (1F), −145.0 ppm (2F) ¹H-NMR (CDCl₃): 5.13 ppm (1H), 5.24 ppm (1H), 7.19 ppm (1H), 8.71 ppm (2H), 8.93 ppm (1H)

### Example 8

### Synthesis of Polymer Having Fluorine-containing Ether Structure in its Side Chain

Into a 50 ml eggplant type glass flask equipped with a stirrer were poured 6.14 g of perfluoro(9,9-dihydro-2,5-bistrifluoromethyl-3,6-dioxanonenoic acid)-(2-aminopyrimidine)amide, 4.14 g of 8.0% by weight perfluorohexane solution of:
[H [symbol] CF₂CF₂ [symbol] ₃COO [symbol] ₂
and 10 ml of HCFC₁₄₁b. After sufficiently replacing the inside of the flask with nitrogen, stirring was carried out at 20° C. for 24 hours in a nitrogen stream. The reaction mixture was poured into a solution mixture of HCFCl41b and hexane of 9:1, followed by separation and drying in vacuo to obtain 1.1 g of a light-yellow polymer.

According to ¹⁹F-NMR, 1H-NMR and IR analyses, the obtained polymer was a fluorine-containing polymer containing only the above-mentioned structural unit of the fluorine-containing ether and has pyrimidine at an end of its side chain. Also the number average molecular weight of the fluorine-containing polymer measured by GPC analysis using tetrahydrofuran (THF) as a solvent was 11,200, and the weight average molecular weight thereof was 17,000. Also as a result of TGA measurement in the air, T_{d10} was 235° C. and T_{d50} was 393° C. This compound was a solid being soluble in acetone.

¹⁹F-NMR (CD₃COCD₃): −75 to −83 ppm (9F), −84 to −87 ppm (1F), −128 ppm (1F), −144 ppm (1F), −166 to −180 ppm (1F) ¹H-NMR (CD₃COCD₃): 2.8 to 3.3 ppm (2H), 7.3 ppm (1H), 8.7 ppm (2H), 11.0 ppm (1H)

### Preparation Example 1

### Synthesis of Polymer Having Fluorine-containing Ether Structure in its Side Chain

Into a 100 ml eggplant type glass flask equipped with a stirrer were poured 29.7 g of perfluoro(9,9-dihydro-2,5-bistrifluoromethyl-3,6-dioxanonenoic acid chloride), 29.2 g of 8.0% by weight perfluorohexane solution of:
[H [symbol] CF₂CF₂ [symbol] ₃COO [symbol] ₂
and 5 ml of HCFC141b. After sufficiently replacing the inside of the flask with nitrogen, stirring was carried out at 20° C. for 24 hours in a nitrogen stream. The obtained liquid having a high viscosity was poured into hexane, followed by separation and drying in vacuo to obtain 24.3 g of a colorless transparent polymer. According to ¹⁹F-NMR, ¹H-NMR and IR analyses, the obtained polymer was a fluorine-containing polymer containing only the above-mentioned structural unit of the fluorine-containing ether and having acid chloride at an end of its side chain. The number average molecular weight of this polymer measured by GPC analysis using tetrahydrofuran (THF) as a solvent was 8,000, and the weight average molecular weight thereof was 12,300.

### Example 9

Into a 200 ml four-necked flask equipped with a thermometer and dropping funnel were poured 15 ml of dehydrated DMF, 1.90 g of 1,2-aminopyrimidine and 3.00 g of triethylamine in a nitrogen atmosphere. Then a solution prepared by dissolving 7.10 g of the polymer of Preparation Example 1 having acid chloride at an end of its side chain in 20 ml of HCFCl41b was slowly added thereto dropwise with stirring at room temperature. After completion of the addition, stirring was carried out overnight at room temperature. The reaction solution subjected to concentration was dissolved in acetone and after re-precipitation with water, was subjected to separation and drying in vacuo to obtain 3.76 g of a light-yellow product. According to ¹⁹F-NMR, ¹H-NMR and IR analyses, it was confirmed that the obtained polymer was the polymer of the present invention. This polymer was a solid. Also as a result of TGA measurement in the air, T_{d10} was 281° C. and T_{d50} was 430° C.

¹⁹F-NMR (CD₃COCD₃): −75 to −83 ppm (9F), −84 to −87 ppm (1F), −128 ppm (1F), −144 ppm (1F), −166 to −180 ppm (1F) ¹H-NMR (CD₃COCD₃): 2.8 to 3.1 ppm (2H), 7.1 ppm (1H), 8.7 ppm (2H), 9.5 ppm (1H)

### Example 10

Into a 200 ml four-necked flask equipped with a thermometer and dropping funnel were poured 35 m of dehydrated DMF, 9.25 g of p-phenylenediamine and 4.6 g of triethylamine in a nitrogen atmosphere. Then a solution prepared by dissolving 11.6 g of the polymer of Preparation Example 1 having acid chloride at an end of its side chain in 20 ml of HCFCl41b was slowly added thereto dropwise with stirring at room temperature. After completion of the addition, stirring was carried out overnight at room temperature. After re-precipitation with water, the reaction solution was subjected to separation and drying in vacuo to obtain 11.7 g of a light-yellow product. According to ¹⁹F-NMR, ¹H-NMR and IR analyses, it was confirmed that the obtained polymer was the polymer of the present invention. This polymer was a solid. Also as a result of TGA measurement in the air, T_{d10} was 340° C. and T_{d50} was 406° C.

¹⁹F-NMR (CD₃COCD₃): −75 to −83 ppm (9F), −84 to −87 ppm (1F), −128 ppm (1F), −144 ppm (1F), −166 to −180 ppm (1F) ¹H-NMR (CD₃COCD₃): 2.8 to 3.1 ppm (2H), 6.7 ppm (2H), 7.2 ppm (2H), 7.6 ppm (2H), 8.1 ppm (1H)

### Example 11

To 0.93 g of perfluoro(9,9-dihydro-2,5-bistrifluoromethyl-3,6-dioxanonenoic acid)-(2-aminoyrimidine)amide prepared in Example 5 was added 1.84 g of trifluoroacetic acid, followed by stirring at room temperature for one day. Excessive trifluoroacetic acid was removed by heating under reduced pressure and a liquid was obtained. As a result of TGA measurement of this liquid in the air, T_{d10} was 220° C. and T_{d50} was 235° C.

### Example 12

To 0.56 g of perfluoro(9,9-dihydro-2,5-bistrifluoromethyl-3,6-dioxanonenoic acid)-(3-(1,2,4-triazole))amide prepared in Example 6 was added 1.40 g of trifluoroacetic acid, followed by stirring at room temperature for one day. Excessive trifluoroacetic acid was removed by heating under reduced pressure and a liquid was obtained. As a result of TGA measurement of this liquid in the air, T_{d10} was 210° C. and T_{d50} was 221° C.

### Example 13

To 1.20 g of perfluoro(9,9-dihydro-2,5-bistrifluoromethyl-3,6-dioxanonenoic acid)-(2-aminoyrimidine)amide prepared in Example 5 was added 0.90 g of trifluoromethanesulfonic acid, followed by stirring at room temperature for one day. Excessive trifluoromethanesulfonic acid was removed by heating under reduced pressure and a liquid was obtained. As a result of TGA measurement of this liquid in the air, T_{d10} was 245° C. and T_{d50} was 263° C.

### Example 14

To 0.97 g of perfluoro(9,9-dihydro-2,5-bistrifluoromethyl-3,6-dioxanonenoic acid)-(3-(1,2,4-triazole))amide prepared in Example 6 was added 1.60 g of trifluoromethanesulfonic acid, followed by stirring at room temperature for one day. Excessive trifluoromethanesulfonic acid was removed by heating under reduced pressure and a liquid was obtained. As a result of TGA measurement of this liquid in the air, T_{d10} was 230° C. and T_{d50} was 241° C.

### Example 15

To 0.97 g of perfluoro(9,9-dihydro-2,5-bistrifluoromethyl-3,6-dioxanonenoic acid)-(4-aminophenyl)amide prepared in Example 4 was added 1.60 g of trifluoromethanesulfonic acid, followed by stirring at room temperature for one day. Excessive trifluoromethanesulfonic acid was removed by heating under reduced pressure and a liquid was obtained. As a result of TGA measurement of this liquid in the air, T_{d10} was 250° C. and T_{d50} was 261° C.

### Comparative Example 1

To 1 g of pyrimidine was added 1 g of trifluoroacetic acid, followed by stirring at room temperature for one day. Excessive trifluoroacetic acid was removed by heating under reduced pressure and a solid was obtained.

### Comparative Example 2

To 1 g of pyrimidine was added 1.8 g of trifluoromethanesulfonic acid, followed by stirring at room temperature for one day. Excessive trifluoromethanesulfonic acid was removed by heating under reduced pressure and a solid was obtained.

### Comparative Example 3

To 1 g of phenylenediamine was added 1.4 g of trifluoromethanesulfonic acid, followed by stirring at room temperature for one day. Excessive trifluoromethanesulfonic acid was removed by heating under reduced pressure and a solid was obtained.

### Example 16

To 1.2 g of the polymer of Example 9 having amidopyrimidine in its side chain was added 1.8 g of trifluoromethanesulfonic acid, followed by stirring at room temperature for one day. Excessive trifluoromethanesulfonic acid was removed by heating under reduced pressure and a liquid was obtained. As a result of TGA measurement of this liquid in the air, T_{d10} was 310° C. and T_{d50} was 430° C.

### Example 17

### Synthesis of diiodide of perfluoro(2,5-bistrifluoromethyl-3,6-dioxanonanoic acid)-(1,3-dimethylpyrimidine-2-yl)amide

Into a 500 ml four-necked flask equipped with a thermometer and dropping funnel were poured 100 m of THF and 30 g of perfluoro(2,5-bistrifluoromethyl-3,6-dioxanonanoic acid)-(2-pyrimidine)amide in a nitrogen atmosphere. Thereto was slowly added dropwise 10 g of methyl iodide on a water bath with stirring. After completion of the addition, stirring was carried out at room temperature for one hour. After completion of the reaction, remaining THF and CH₃I were removed under reduced pressure to obtain 45 g of diiodide of perfluoro(2,5-bistrifluoromethyl-3,6-dioxanonanoic acid)-(1,3-dimethylpyrimidine-2-yl)amide. According to ¹⁹F-NMR and ¹H-NMR analyses, it was confirmed that the obtained product was the above-mentioned compound. This compound was a liquid.

¹⁹F-NMR (CD₃COCD₃): −78 to −80 ppm (7F), −82 to −85 ppm (4F), −92 ppm (2F), −115 ppm (2F), −132 ppm (1F), −145.0 ppm (1F) ¹H-NMR (CD₃COCD₃): 4.1 to 4.5 ppm (6H), 7.2 ppm (1H), 8.7 ppm (2H), 9.0 ppm (1H)

### Example 18

### Synthesis of (2-pyrimidine)amide Having Long Chain Fluorine-containing Ether

Into a 500 ml four-necked flask equipped with a thermometer and dropping funnel were poured 150 m of dehydrated DMF, 10 g of aminopyrimidine and 10 g of triethylamine in a nitrogen atmosphere. Thereto was slowly added dropwise 120 g of the following acid fluoride:
C₃F₇O—(CF₂CF₂CF₂O)ₙ—CF₂CF₂COF
(n is 10 on the average) containing a long chain fluorine-containing ether and having an average molecular weight of 2,000 on an ice bath with stirring. After completion of the addition, the mixture temperature was gradually increased to room temperature and stirring was carried out at room temperature for one hour. Then the reaction solution was subjected to separation with an acid and water to take out an oil layer. By concentration of the oil layer, 122 g of the following (2-pyrimidine)amide:
C₃F₇O—(CF(CF₃) CF₂O)₁₂—CF(CF₃)CONH—(2-pyrimidine)
having a long chain fluorine-containing ether was obtained. According to ¹⁹F-NMR and 1H-NMR analyses, it was confirmed that the obtained product was the above-mentioned compound. This compound was insoluble in acetone and ethyl acetate and was a liquid at room temperature. Also as a result of TGA measurement in the air, T_{d10} was 310° C. and T_{d50} was 328° C.

¹H-NMR (CD₃COCD₃): 7.2 ppm (1H), 8.7 ppm (2H), 9.0 ppm (1H)

### Experimental Example 1

(Effect as an Acid-removing Agent)

Into 20 ml of 1 N aqueous solution of hydrochloric acid was poured 30 g of (2-pyrimidine)amide having a long chain fluorine-containing ether prepared in Example 18, followed by stirring at room temperature for 0.5 hour. During the stirring, the solution was separated into two layers. Also after completion of the stirring, the solution was separated into two layers, namely a lower layer of the (2-pyrimidine)amide having a long chain fluorine-containing ether and an upper layer of the aqueous solution of hydrochloric acid. According to the investigation of acidity of this aqueous solution by using a pH test paper, the solution was changed from strong acid to neutral by treating with the amide.

### Experimental Example 2

(Effect as an Acid-removing Agent)

Into 20 ml of 1 N aqueous solution of acetic acid was poured 30 g of (2-pyrimidine)amide having a long chain fluorine-containing ether prepared in Example 18, followed by stirring at room temperature for 0.5 hour. During the stirring, the solution was separated into two layers. Also after completion of the stirring, the solution was separated into two layers, namely a lower layer of the (2-pyrimidine)amide having a long chain fluorine-containing ether and an upper layer of the aqueous solution of acetic acid. According to the investigation of acidity of this aqueous solution by using a pH test paper, the solution was changed from acid to neutral by treating with the amide.

### Experimental Example 3

(Confirmation of Lubricity)

On a polystyrene film of about 100 μm thick×3 cm×10 cm was coated metallic cobalt by vacuum vapor deposition, and further thereon was formed an about 6 μm thick polystyrene layer by spin coating. On this layer was coated perfluoro(2,5-bistrifluoromethyl-3,6-dioxadodecanoic acid (lubrication layer I) or a liquid composition prepared by mixing 10 g of perfluoro(2,5-bistrifluoromethyl-3,6-dioxadodecanoic acid)-(2-pyrimidine)amide and 10 g of perfluoro(2,5-bistrifluoromethyl-3,6-dioxadodecanoic acid (lubrication layer II) as a lubrication layer by spin coating. The coating amount was about 10 mg/m². The coated film was set so that the lubrication layer was placed down, and was rubbed 100 times from below with a rod having a tip in the form of semi-cylindrical section of 5 cm long×3 cm diameter by applying a force of about 10 gf. A frictional force and remaining amount of metallic cobalt at the first rubbing and 100th rubbing are shown in Table 1.

A change in the frictional force is evaluated by a speed of rubbing when rubbing with a given force, and the remaining amount of metallic cobalt is evaluated by observing the surface of the coated layer with an optical microscope and checking an amount of falling cobalt (when adhesion is not good, cobalt powder falls). With respect to the evaluation criteria, when the rubbing speed does not change greatly, it is assumed to be ◯ and when the rubbing speed decreases greatly, it is assumed to be ×. Further when an amount of falling cobalt powder is small, it is assumed to be ◯ and when an amount of falling cobalt powder is large, it is assumed to be ×.

TABLE 1

| | | Amount of | | Amount of |
|---|---|---|---|---|
| | Rubbing | remaining | Rubbing | remaining |
| Lubrication | speed | cobalt | speed | cobalt |
| layer | (1st) | (1st) | (100th) | (100th) |
| Nil | X | ◯ | X | X |
| I | ◯ | ◯ | X | X |
| II | ◯ | ◯ | ◯ | ◯ |

### Experimental Example 4

(Confirmation of Actuator Effect)

A film of 0.2 mm thick×1 cm×5 cm of NAFFION 117 (trademark of DuPont) was dipped in a 10⁻² N aqueous solution of AU(III)(phenanthrene)Cl3 at room temperature for 10 hours and then washed with pure water, and those procedures were repeated five times. Then the film was dipped in a 10⁻³ N aqueous solution of Na₂SO₃ at 50° C. for eight hours and thus a composite film of Au and NAFFION was obtained. Subsequently this composite film was dipped at room temperature for 12 hours in a 1 N aqueous solution of LiCl, 1 N aqueous solution of NH₄Cl or 1 N ethanol solution of the amide compound prepared in Example 1 to carry out ion exchanging. As a result, a NAFFION film in which a counter ion of sulfonic acid is Li⁺ (counter cation I), a NAFFION film in which a counter ion of sulfonic acid is NH₄⁺ (counter cation II) and a NAFFION film in which a counter ion of sulfonic acid is a cation derived from the amide compound prepared in Example 1 (counter cation III) were obtained.

Next, a tip of each film was nipped with a Pt foil, an electric wire was connected to the Pt foil and the film was connected to a potentiostat (HA-501G available from Hokuto Denko Kabushiki Kaisha) through the wire. The film was then dipped in water and a potential of 3V was applied. The degree of deformation was observed with naked eyes. The results are shown in Table 2.

In the evaluation, the degree of deformation was classified into three stages of ◯, Δ and ×.

TABLE 2

| Counter cation | Degree of deformation |
|---|---|
| I | Δ |
| II | Δ |
| III | ◯ |

### Preparation Example 2

### Synthesis of Polymer Having Fluorine-containing Ether Structure in its Side Chain

Into a 100 ml eggplant type glass flask equipped with a stirrer were poured 12 g of perfluoro(12,12-dihydro-2,5,8-tristrifluoromethyl-3,6,9-trioxadodecenoic acid chloride): 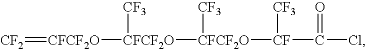
10.4 g of perfluoro(12,12,2-trihydro-2,5,8-tristrifluoromethyl-3,6,9-trioxadodecane: 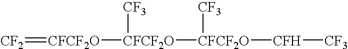
and then 36 g of 8.0% by weight perfluorohexane solution of:
[H [symbol] CF₂CF₂ [symbol] ₃COO [symbol] ₂
and 20 ml of HCFCl41b. After sufficiently replacing the inside of the flask with nitrogen, stirring was carried out at 20° C. for 24 hours in a nitrogen stream. The obtained liquid having a high viscosity was poured into hexane, followed by separation and drying in vacuo to obtain 20.5 g of a colorless transparent polymer. According to ¹⁹F-NMR, ¹H-NMR and IR analyses of the obtained polymer, a percent by mole ratio of a unit having an acid chloride at an end of its side chain to a unit having no functional group at an end of its side chain was 53/47. The number average molecular weight of this polymer measured by GPC analysis using tetrahydrofuran (THF) as a solvent was 6,800, and the weight average molecular weight thereof was 8,300.

### Example 19

Into a 200 ml four-necked flask equipped with a thermometer and dropping funnel were poured 15 m of dehydrated DMF, 2.1 g of 2-aminopyrimidine and 5.00 g of triethylamine in a nitrogen atmosphere. Then a solution prepared by dissolving 10 g of the polymer of Preparation Example 2 having acid chloride at an end of its side chain in 20 ml of HCFCl41b was slowly added thereto dropwise with stirring at room temperature. After completion of the addition, stirring was carried out overnight at room temperature. The reaction solution subjected to concentration was dissolved in acetone and after re-precipitation with water, was subjected to separation and drying in vacuo to obtain 9.1 g of a light-yellow product. According to ¹⁹F-NMR, ¹H-NMR and IR analyses, it was confirmed that the obtained product was the polymer of the present invention. This polymer was a solid. Also as a result of TGA measurement in the air, T_{d10} was 251° C. and T_{d50} was 380° C.

¹⁹F-NMR (CD₃COCD₃): −75 to −83 ppm (10.4F), −84 to −87 ppm (1F), −128 ppm (1F), −144 ppm (1F), −166 to −180 ppm (1.5F) ¹H-NMR (CD₃COCD₃): 2.8 to 3.1 ppm (4.9H), 7.1 ppm (1.1H), 8.7 ppm (2.1H), 9.5 ppm (1.1H)

### Preparation Example 3

### Synthesis of Polymer Having Fluorine-containing Ether Structure in its Side Chain

Into a 100 ml eggplant type glass flask equipped with a stirrer were poured 8 g of perfluoro(12,12-dihydro-2,5,8-tristrifluoromethyl-3,6,9-trioxadodecenoic acid chloride): 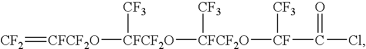
20.7 g of perfluoro(12,12,2-trihydro-2,5,8-tristrifluoromethyl-3,6,9-trioxadodecane: 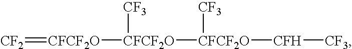
49 g of 8.0% by weight perfluorohexane solution of:
[H [symbol] CF₂CF₂ [symbol] ₃COO [symbol] ₂
and 28 ml of HCFC141b. After sufficiently replacing the inside of the flask with nitrogen, stirring was carried out at 20° C. for 24 hours in a nitrogen stream. The obtained liquid having a high viscosity was poured into hexane, followed by separation and drying in vacuo to obtain 25 g of a colorless transparent polymer. According to ¹⁹F-NMR, ¹H-NMR and IR analyses of the obtained polymer, a percent by mole ratio of a unit having an acid chloride at an end of its side chain to a unit having no functional group at an end of its side chain was 24/76. The number average molecular weight of this polymer measured by GPC analysis using tetrahydrofuran (THF) as a solvent was 6,200, and the weight average molecular weight thereof was 7,600.

### Example 20

Into a 200 ml four-necked flask equipped with a thermometer and dropping funnel were poured 15 ml of dehydrated DMF, 1.5 g of 2-aminopyrimidine and 5.00 g of triethylamine in a nitrogen atmosphere. Then a solution prepared by dissolving 10 g of the polymer of Preparation Example 3 having acid chloride at an end of its side chain in 20 ml of HCFCl41b was slowly added thereto dropwise with stirring at room temperature. After completion of the addition, stirring was carried out overnight at room temperature. The reaction solution subjected to concentration was dissolved in acetone and after re-precipitation with water, was subjected to separation and drying in vacuo to obtain 8.7 g of a light-yellow product. According to ¹⁹F-NMR, ¹H-NMR and IR analyses, it was confirmed that the obtained product was the polymer of the present invention. This polymer was a solid. Also as a result of TGA measurement in the air, T_{d10} was 264° C. and T_{d50} was 360° C.

¹⁹F-NMR (CD₃COCD₃): −75 to −83 ppm (11.2F), −84 to 31 87 ppm (1F), −128 ppm (1F), −144 ppm (1F), −166 to −180 ppm (1.7F) ¹H-NMR (CD₃COCD₃): 2.8 to 3.1 ppm (5.4H), 7.1 ppm (0.5H), 8.7 ppm (1.0H), 9.5 ppm (0.5H)

### Example 21

### Synthesis of 2,4,6-tri{perfluoro(2,5,8-tristrifluoromethyl-3,6,9-trioxadodecanoyl)}-1,3,5-[triazine]

Into a 100 ml autoclave were poured 35 g of perfluoro(2,5,8-tristrifluoromethyl-3,6,9-trioxadodecanonitrile): 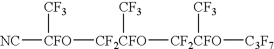
and 0.2 g of silver oxide, and after replacing the inside of the autoclave with nitrogen, heating was carried out at 140° C. for 18 hours with stirring. After completion of the reaction, silver oxide was removed by filtration, and by distilling off the filtrate by heating in vacuo, 20 g of 2,4,6-tri{perfluoro(2,5,8-tristrifluoromethyl-3,6,9-trioxadodecanoyl) }-1,3,5-[triazine]: 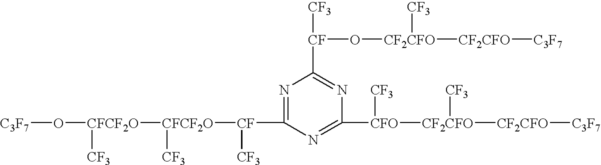
was obtained. According to ¹⁹F-NMR, ¹H-NMR and IR analyses, it was confirmed that the obtained product was the above-mentioned compound. This compound was a liquid at room temperature. Also as a result of TGA measurement in the air, T_{d10} was 160° C. and T_{d50} was 165° C.

¹⁹F-NMR (CD₃COCD₃): −78 to −80 ppm (1 F), −82 to −85 ppm (5F), −97 ppm (2F), −118 ppm (2F), −132 ppm (1F), −147 ppm (2F) ¹H-NMR (CD₃COCD₃): There is no peak. IR: 1,620 cm⁻¹ (C═N)

### Example 22

### Synthesis of 2,4-bis{perfluoro(2,5,8-tristrifluoromethyl-3,6,9-trioxadodecanoyl)}-1,3,5-[triazine]-6-ol

Into 30 ml of sulfolane was poured 0.57 g of sodium hydride, and thereto were added dropwise 30 g of perfluoro(2,5,8-tristrifluoromethyl-3,6,9-trioxadodecanonitrile) and 1.4 g of urea on a water bath. After completion of the addition, stirring was carried out for one hour, followed by heating with stirring at 80° C. for eight hours. After completion of the reaction, washing with water and separation were carried out to take out an oil layer. By distilling off the unreacted product by heating in vacuo, 18 g of 2,4-bis{perfluoro(2,5,8-tristrifluoromethyl-3,6,9-trioxadodecanoyl) }-1,3,5-[triazine]-6-ol: 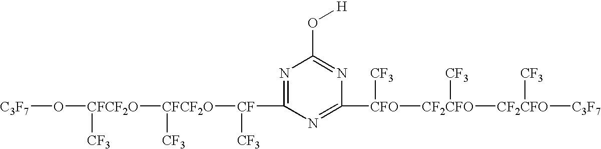
was obtained. According to 1⁹F-NMR, ¹H-NMR and IR analyses, it was confirmed that the obtained product was the above-mentioned compound. This compound was soluble in acetone, ethyl acetate, etc. and was a liquid at room temperature. Also as a result of TGA measurement in the air, T_{d10} was 170° C. and T_{d50} was 175° C.

¹⁹F-NMR (CD₃COCD₃): −78 to −80 ppm (11F), −82 to −85 ppm (5F), −97 ppm (2F), −118 ppm (2F), −132 ppm (1F), −147 ppm (2F) ¹H-NMR (CD₃COCD₃): There is no peak. IR: 1,620 cm⁻¹ (C═N), 3,200 cm⁻¹ (OH)

### Example 23

### Synthesis of 2,4-diamino-6-{perfluoro(2,5,8-tristrifluoromethyl-3,6,9-trioxadodecanoyl)}-1,3,5-[triazine]

Into 30 ml of sulfolane was poured 0.7 g of sodium hydride, and thereto were added dropwise 30 g of perfluoro(2,5,8-tristrifluoromethyl-3,6,9-trioxadodecanonitrile) and 4.3 g of dicyandiamide on a water bath. After completion of the addition, stirring was carried out for one hour, followed by heating with stirring at 80° C. for eight hours. After completion of the reaction, washing with ammonia water and separation were carried out to take out an oil layer. By distilling off the unreacted product by heating in vacuo, 21 g of 2,4-diamino-6-{perfluoro(2,5,8-tristrifluoromethyl-3,6,9-trioxadodecanoyl)} -1,3,5-[triazine]: 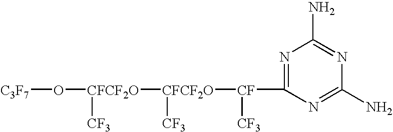
was obtained. According to ¹⁹F-NMR, ¹H-NMR and IR analyses, it was confirmed that the obtained product was the above-mentioned compound. This compound was soluble in acetone, ethyl acetate, etc. and was a solid at room temperature.

¹⁹F-NMR (CD₃COCD₃): −78 to −80 ppm (11F), −82 to −85 ppm (5F), −97 ppm (2F), −118 ppm (2F), −132 ppm (1F), −147 ppm (2F) ¹H-NMR (CD₃COCD₃): There is no peak. IR: 1,620 cm⁻¹ (C═N), 3,300 cm⁻¹ (NH₂)

### Preparation Example 4

### Synthesis of Polymer Having Fluorine-containing Ether Structure in its Side Chain

Into a 100 ml eggplant type glass flask equipped with a stirrer were poured 9.8 g of perfluoro(12,12-dihydro-2,5,8-tristrifluoromethyl-3,6,9-trioxadodecanonitrile): 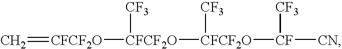
10.2 g of perfluoro(12,12,2-trihydro-2,5,8-tristrifluoromethyl-3,6,9-trioxadodecane: 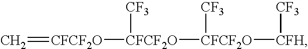
24 g of 8.0% by weight perfluorohexane solution of:
[H [symbol] CF₂CF₂ [symbol] ₃COO [symbol] ₂
and 20 g of HCFC141b. After sufficiently replacing the inside of the flask with nitrogen, stirring was carried out at 20° C. for 24 hours in a nitrogen stream. The obtained liquid having a high viscosity was poured into hexane, followed by separation and drying in vacuo to obtain 16.0 g of a colorless transparent polymer. According to ¹⁹F-NMR, ¹H-NMR and IR analyses of the obtained polymer, a percent by mole ratio of a unit having cyano group at an end of its side chain to a unit having no functional group at an end of its side chain was 48/52.

### Example 24

Into a 200 ml three-necked flask equipped with a stirrer, thermometer and cooling tube were poured 10 g of the polymer having cyano group at an end of its side chain obtained in Preparation Example 4, 50 g of N,N′-dimethylformamide, 0.9 g of dicyandiamide and 0.8 g of potassium hydride. This solution of the mixture was heated up to 98° C. and stirred for six hours. The reaction solution was dissolved in acetone, and after re-precipitation with water, was subjected to separation and drying in vacuo to obtain 9.4 g of a brown product. According to ¹⁹F-NMR, ¹H-NMR and IR analyses, it was confirmed that the nitrile group of the polymer had been converted to the following diaminotriazine.

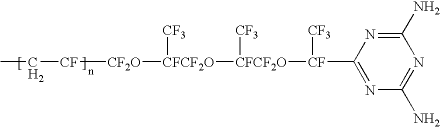

### Preparation Example 5

### Synthesis of Polymer Having Fluorine-containing Ether Structure in its Side Chain

Into a 100 ml eggplant type glass flask equipped with a stirrer were poured 14.9 g of perfluoro(12,12-dihydro-2,5,8-tristrifluoromethyl-3,6,9-trioxadodecanonitrile): 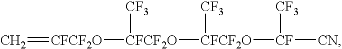
5.2 g of perfluoro(12,12,2-trihydro-2,5,8-tristrifluoromethyl-3,6,9-trioxadodecane: 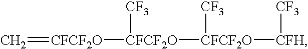
24 g of 8.0% by weight perfluorohexane solution of:
[H [symbol] CF₂CF₂ [symbol] ₃COO [symbol] ₂
and 20 g of HCFCl41b. After sufficiently replacing the inside of the flask with nitrogen, stirring was carried out at 20° C. for 24 hours in a nitrogen stream. The obtained liquid having a high viscosity was poured into hexane, followed by separation and drying in vacuo to obtain 17.3 g of a colorless transparent polymer. According to ¹⁹F-NMR, ¹H-NMR and IR analyses of the obtained polymer, a percent by mole ratio of a unit having cyano group at an end of its side chain to a unit having no functional group at an end of its side chain was 83/17.

### Example 25

Into a 100 ml three-necked flask equipped with a stirrer, thermometer and cooling tube were poured 9.0 g of the polymer having cyano group at an end of its side chain obtained in Preparation Example 5, 45 g of sulfolane, 0.5 g of dicyandiamide and 0.5 g of potassium hydride. This solution of the mixture was heated up to 98° C. and stirred for three hours. The reaction solution was dissolved in acetone, and after re-precipitation with water, was subjected to separation and drying in vacuo to obtain 7.0 g of a brown product. According to ¹⁹F-NMR, ¹H-NMR and IR analyses, it was confirmed that the cyano group of the polymer had been converted to diaminotriazine. This polymer was a viscous liquid.

¹⁹F-NMR (CD₃COCD₃): −77 to −82 ppm (39.0F), −82 to −85 ppm (14.6F), −120 to −121 ppm (1F), -121 to −123 ppm (2F), −128 to −129 ppm (1.2F), −137 to −138 ppm (1F), −142 to −145 ppm (7.8F), −146 to −147 ppm (3.1F) ¹H-NMR (CD₃COCD₃): 2.5 to 3.3 ppm (8.4H), 6.5 to 6.8 ppm (1H), 6.8 to 7.3 ppm (4.0H)

### INDUSTRIAL APPLICABILITY

The present invention can provide an ionic liquid type functional material which is excellent in oxidation resistance, ion stability, heat resistance, lubricity and water insolubility and low in a viscosity and is useful for a lubricant, acid-removing agent, various ionic liquid materials or solid materials, electrolyte for solar cell and actuator material.

1. An ionic liquid type functional material comprising an aromatic compound which has a fluorine-containing ether chain and is represented by the formula (1):
[Ra-D [symbol] ₘRy (1)
wherein -D- is a fluoroether unit represented by the formula (1-1): [symbol]
O—R [symbol] ₙ or [symbol] R—O [symbol] ₙ (1-1)
in which R is at least one selected from divalent fluorine-containing alkylene groups having 1 to 5 carbon atoms in which at least one of hydrogen atoms is replaced by fluorine atom; n is an integer of from 1 to 20, and when m is not less than 2, two or more of D may be the same or different;
Ra is a monovalent organic group which has 1 to 20 carbon atoms and does not contain said D, and when m is not less than 2, two or more of Ra may be the same or different; m is an integer of from 1 to 4;
Ry is a mono-, di-, tri- or tetra-valent organic group having 2 to 30 carbon atoms which has at least one selected from basic functional groups Y¹ and/or salts Y² of the basic functional groups and contains an aromatic ring structure,
provided that a unit of—O—O— is not contained in said formulae (1) and (1-1).

2. The ionic liquid type functional material of claim 1, wherein in said formula (1), —O—R— in -D- has at least one kind of fluoroether unit selected from the group consisting of —(OCFZ¹CF₂)—, —(OCF₂CF₂CF₂)—, —(OCH₂CF₂CF₂)—, —(OCFZ²)—, —(OCZ³₂)—, —(CFZ¹CF₂O)—, —(CF₂CF₂CF₂O)—, —(CH₂CF₂CF₂O)—, —(CFZ²O)— and —(CZ³₂O)—, wherein Z¹ and Z² are the same or different and each is H, F or CF₃; Z³ is CF₃.

3. The ionic liquid type functional material of claim 1, wherein Ra is selected from fluorine-containing alkyl groups Rx having 1 to 20 carbon atoms.

4. The ionic liquid type functional material of claim 1, wherein Ra is a monovalent organic group Ry′ having 2 to 20 carbon atoms which has at least one selected from the basic functional groups Y¹ and/or the salts Y² of the basic functional groups and contains an aromatic ring structure.

5. The ionic liquid type functional material of claim 1, wherein the basic functional group or the salt of the basic functional group contained in said Ry is at least one kind selected from amines, imines, enamines, ketimines, azines and salts thereof.

6. An ionic liquid type functional material comprising a fluorine-containing polymer represented by the formula (M-1):
-(M1)-(A1)- (M-1)
wherein the structural unit M1 is at least one selected from structural units derived from ethylenic monomers having, in a side chain thereof, a moiety represented by the formula (2):
-D¹-Ry¹ (2)
in which -D¹- is a fluoroether unit represented by the formula (2-1): [symbol]
O—R¹ [symbol] ₙ₁ or [symbol] R¹—O [symbol] ₙ₁ (2-1)
wherein R¹ is at least one selected from divalent fluorine-containing alkylene groups having 1 to 5 carbon atoms in which at least one of hydrogen atoms is replaced by fluorine atom; n1 is an integer of from 1 to 20; Ry¹ is a monovalent organic group having 2 to 30 carbon atoms which has at least one selected from basic functional groups Y¹ and/or salts Y² of the basic functional groups and contains an aromatic ring structure, provided that a unit of —O—O— is not contained in the structural unit M1 and the formula (2-1); the structural unit A1 is a structural unit derived from a monomer being copolymerizable with the monomer being capable of providing the structural unit M1, and the structural units M1 and A1 are contained in amounts of from 1 to 100% by mole and from 0 to 99% by mole, respectively.

7. An ionic liquid type functional material comprising a fluorine-containing polymer represented by the formula (M-2):
-(M2)-(A2)- (M-2)
wherein the structural unit M2 is a structural unit derived from an ethylenic monomer having, in its side chain, a moiety represented by the formula (3):
-Ry² [symbol] D¹-Ra¹]ₘ₁ (3)
in which Ry² is a di-, tri- or tetra-valent organic group having 2 to 30 carbon atoms which has at least one of basic functional groups Y¹ and/or salts Y² of the basic functional groups and contains an aromatic ring structure; Ra¹ is a monovalent organic group which has 1 to 20 carbon atoms and does not contain D¹, and when m1 is not less than 2, two or more of Ra¹ may be the same or different; m1 is an integer of from 1 to 3; -D¹- is selected from the same units as defined in the formula (2) of claim 6, and when m1 is not less than 2, two or more of D may be the same or different, provided that a unit of —O—O— is not contained in the structural unit M2 and the formulae (2-1); the structural unit A2 is a structural unit derived from a monomer being copolymerizable with the monomer being capable of providing the structural unit M2, and the structural units M2 and A2 are contained in amounts of from 1 to 100% by mole and from 0 to 99% by mole, respectively.

8. The ionic liquid type functional material of claim 7, wherein Ra¹ is selected from fluorine-containing alkyl groups Rx¹ having 1 to 20 carbon atoms.

9. The ionic liquid type functional material of claim 6, wherein the basic functional group Y¹ or the salt Y² of the basic functional group contained in said Ry¹ is at least one kind selected from amines, imines, enamines, ketimines, azines and salts thereof.

10. An aromatic compound which has a fluorine-containing ether chain and is represented by the formula (4):
[Rx²-D² [symbol] ₘ₂Ry³ (4)
wherein -D²- is a fluoroether unit represented by the formula (4-1): [symbol]
O—R² [symbol] ₙ₂ or [symbol] R²—O [symbol] ₙ₂ (4-1)
in which R² is at least one selected from divalent fluorine-containing alkylene groups having 1 to 5 carbon atoms in which at least one of hydrogen atoms is replaced by fluorine atom; n2 is an integer of from 1 to 20, and when m2 is not less than 2, two or more of D² may be the same or different; Ry³ is a mono-, di-, tri- or tetra-valent organic group having 2 to 30 carbon atoms which has at least one of amines and/or salts of amines and contains an aromatic ring structure; Rx² is a fluorine-containing alkyl group having 1 to 20 carbon atoms, and when m2 is not less than 2, two or more of Rx² may be the same or different; m2 is an integer of from 1 to 4, provided that a unit of —O—O— is not contained in said formulae (4) and (4-1).

11. An aromatic compound which has a fluorine-containing ether chain and is represented by the formula (5):
CX¹X²═CX³—(CX⁴X⁵)ₙ₃(C═O)ₙ₄-D-Ry⁴ (5)
wherein X¹, X², X⁴ and X⁵ are the same or different and each is hydrogen atom or fluorine atom; X³ is selected from hydrogen atom, fluorine atom, CH₃ and CF₃; n3 and n4 are the same or different and each is 0 or 1; Ry⁴ is a monovalent organic group having 2 to 30 carbon atoms which has at least one of amines and/or salts of amines and contains an aromatic ring structure; D² is as defined in the formula (4) of claim 10.

12. The aromatic compound of claim 11 which has a fluorine-containing ether chain and is represented by the formula (6):
CX¹X²═CX³—(CX⁴X⁵)ₙ₃-D²-Ry⁴ (6)
wherein X¹, X², X³, X⁴, X⁵, n3, D² and Ry⁴ are as defined in said formula (5).

13. A fluorine-containing polymer which has a number average molecular weight of from 500 to 1,000,000 and is represented by the formula (M-3):
-(M3)-(A3)- (M-3)
wherein the structural unit M3 is a structural unit represented by the formula (7): 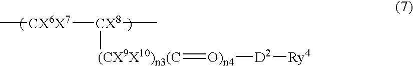
wherein X⁶, X⁷, X⁹ and X¹⁰ are the same or different and each is hydrogen atom or fluorine atom; X⁸ selected from hydrogen atom, fluorine atom, CH₃ and CF₃; n3 and n4 ate the same or different and each is 0 or 1; D² and Ry⁴ are as defined in the formula (5) of claim 11; the structural unit A3 is a structural unit derived from a monomer being copolymerizable with the monomer being capable of providing the structural unit M3, and the structural units M3 and A3 are contained in amounts of from 1 to 100% by mole and from 0 to 99% by mole, respectively.

14. The fluorine-containing polymer of claim 13, wherein the structural unit M3 is a structural unit represented by the formula (8): 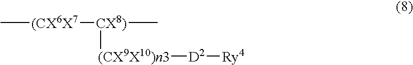
wherein X⁶, X⁷, X⁸, X⁹, X¹⁰, n3, D² and Ry⁴ are as defined in the formula (7).

15. The ionic liquid type functional material of claim 2, wherein Ra is selected from fluorine-containing alkyl groups Rx having 1 to 20 carbon atoms.

16. The ionic liquid type functional material of claim 2, wherein Ra is a monovalent organic group Ry′ having 2 to 20 carbon atoms which has at least one selected from the basic functional groups Y¹ and/or the salts Y² of the basic functional groups and contains an aromatic ring structure.

17. The ionic liquid type functional material of claim 2, wherein the basic functional group or the salt of the basic functional group contained in said Ry is at least one kind selected from amines, imines, enamines, ketimines, azines and salts thereof.

18. The ionic liquid type functional material of claim 3, wherein the basic functional group or the salt of the basic functional group contained in said Ry is at least one kind selected from amines, imines, enamines, ketimines, azines and salts thereof.

19. The ionic liquid type functional material of claim 15, wherein the basic functional group or the salt of the basic functional group contained in said Ry is at least one kind selected from amines, imines, enamines, ketimines, azines and salts thereof.

20. The ionic liquid type functional material of claim 7, wherein the basic functional group Y¹ or the salt Y² of the basic functional group contained in said Ry² is at least one kind selected from amines, imines, enamines, ketimines, azines and salts thereof.
